(19) United States
(12) Patent Application Publication
(10) Pub. No.: US 2018/0094304 A1
(43) Pub. Date: Apr. 5, 2018

(54) USE OF GENETIC PROFILING FOR PREDICTION OF WINE TASTE PREFERENCES AND WINE SELECTION

(71) Applicants: Ron A. Andrews, Coto de Caza, CA (US); Shannon M. Kieran, Scottsdale, AZ (US); Sara Riordan, Roseville, CA (US); Elissa Levin, San Francisco, CA (US); Zack Miskel, Napa, CA (US); Josh Riggs, Trabuco Canyon, CA (US)

(72) Inventors: Ron A. Andrews, Coto de Caza, CA (US); Shannon M. Kieran, Scottsdale, AZ (US); Sara Riordan, Roseville, CA (US); Elissa Levin, San Francisco, CA (US); Zack Miskel, Napa, CA (US); Josh Riggs, Trabuco Canyon, CA (US)

(73) Assignee: Vinome, HEALDSBURG, CA (US)

(21) Appl. No.: 15/671,161

(22) Filed: Aug. 8, 2017

Related U.S. Application Data
(60) Provisional application No. 62/371,842, filed on Aug. 8, 2016.

(51) Int. Cl. C12Q 1/68

(52) U.S. Cl. CPC C12Q 1/6827

(57) ABSTRACT

Methods, compositions, computer systems, and kits for identifying wine taste preferences of individuals are disclosed. In particular, the invention relates to the use of genetic profiling of an individual in combination with questioning about personal characteristics and taste preferences to predict individual wine taste preferences. The methods of the invention can be used for selection of wines that have a high probability of meeting the taste preferences of an individual.

### RELATED APPLICATIONS

This application claims priority to U.S. Provisional Patent Application Ser. No. 62/371,842, filed on Aug. 8, 2016, which is hereby incorporated by reference in its entirety.

### FIELD OF THE INVENTION

The present invention pertains to the wine industry and methods of wine selection. In particular, the invention relates to the use of genetic profiling to predict individual wine taste preferences and methods for selection of wines for an individual based on such genetic profiling.

### BACKGROUND OF THE INVENTION

In the wine industry, it is desired to select wines that meet an individual's taste preferences. However, the large number of wines available and their great variation in taste present a challenge and create uncertainty regarding which wines will best suit the taste preferences of an individual. Moreover, the expansion of the wine industry has been accompanied by increasing numbers of wineries, brands, and wine styles and is making ever more varieties of wines available to the consumer.

Wines have different taste characteristics depending on the type of wine, region where the wine was produced, the age of the wine, and the year the wine was produced. Wine ranking systems devised by professionals in the industry use expert wine tasters in an attempt to provide some guidance to wine consumers, but individual taste preferences vary, and wine rankings may not coincide with whether an individual likes a particular wine. Customers are often unfamiliar with particular wines and unsure of which wines they will like. If customers purchase a wine they do not like, they may feel they have had a bad buying experience with a particular type of wine or a particular winery, restaurant, or store. Sometimes, in order to avoid bad experiences, individuals identify a particular wine they like, and decide they do not feel comfortable trying other wines.

Thus, there remains a need in the wine industry for better methods of determining individual wine taste preferences to allow selection of wines that will satisfy the taste preferences of an individual.

### SUMMARY OF THE INVENTION

The present invention relates to the use of genetic profiling in combination with questioning about personal characteristics and taste preferences to predict individual wine taste preferences. The methods of the invention can be used to provide wines to consumers based on their predicted taste preferences.

In some embodiments, the present invention provides a method for assaying a sample from a subject, the method comprising: a) obtaining a biological sample comprising nucleic acids from a subject; b) processing the sample to isolate or enrich the sample for the nucleic acids; and c) assaying a plurality of SNPs in the sample, wherein the plurality of SNPs comprises one or more SNPs selected from Table 1.

In other embodiments, the invention provides a method comprising: a) obtaining taste preference information from a subject; b) obtaining a biological sample comprising nucleic acids from the subject; c) processing the sample to isolate or enrich the sample for the nucleic acids; and d) analyzing the genotype of the biological sample at a plurality of SNPs in a panel of SNPs identified as useful for identifying taste preference. In some embodiments, the plurality of SNPs is selected from the group consisting of rs838133, rs1421085, rs3930459, rs2274333, rs111615792, rs72921001, rs7792845, rs10772420, rs846672, rs9295791, rs6591536, rs1761667, rs10772397, rs586965, rs5020278, rs11988795, rs4595035, rs12226920, rs1524600, rs61729907, rs1548803, rs4684677, rs42451, rs35680, rs34911341, rs696217, rs26802, rs2708377, rs28757581, rs35744813, rs4920566, rs3935570, rs2277675, rs71443637, rs9276975, rs34160967, rs307377, rs713598, rs1726866, rs10246939, and rs846664. In some embodiments, the plurality of SNPs comprises rs3930459, rs42451, rs2708377, rs71443637, and rs12226920. In some embodiments, the plurality of SNPs comprises rs3930459 and rs42451. In some embodiments, the plurality of SNPs comprises rs3930459 and rs2708377. In some embodiments, the plurality of SNPs comprises rs3930459 and rs71443637. In some embodiments, the plurality of SNPs comprises rs3930459 and rs12226920. In some embodiments, the plurality of SNPs comprises rs42451 and rs2708377. In some embodiments, the plurality of SNPs comprises rs42451 and rs71443637. In some embodiments, the plurality of SNPs comprises rs42451 and rs12226920. In some embodiments, the plurality of SNPs comprises rs2708377 and rs71443637. In some embodiments, the plurality of SNPs comprises rs2708377 and rs12226920. In some embodiments, the plurality of SNPs comprises rs71443637 and rs12226920. In some embodiments, the plurality of SNPs comprises at least 5, 10, 15, 20, 25, 30, 35 or 41 SNPs selected from Table 1. In some embodiments, the method further comprises predicting wine preferences for the subject.

In one embodiment, the present invention provides a method comprising: a) obtaining a biological sample from a subject; b) isolating nucleic acids from the sample; and c) detecting the presence of a plurality of SNPs selected from the group consisting of rs838133, rs1421085, rs3930459, rs2274333, rs111615792, rs72921001, rs7792845, rs10772420, rs846672, rs9295791, rs6591536, rs1761667, rs10772397, rs586965, rs5020278, rs11988795, rs4595035, rs12226920, rs1524600, rs61729907, rs1548803, rs4684677, rs42451, rs35680, rs34911341, rs696217, rs26802, rs2708377, rs28757581, rs35744813, rs4920566, rs3935570, rs2277675, rs71443637, rs9276975, rs34160967, rs307377, rs713598, rs1726866, rs10246939, and rs846664. In some embodiments, the plurality of SNPs comprises at least 2, 3, 4, 5, 10, 15, 20, 25, 30, 35 or 41 SNPs selected from Table 1.

In one embodiment, the present invention provides a method comprising: a) obtaining a biological sample from a subject; b) isolating nucleic acids from the sample; and c) detecting the presence of a plurality of targets selected from the group consisting of FGF21, FTO, OR10A6, OR10A2, OR10A4, OR10A3, OR6A2, CA6, TAS1R3, GNAT3, TAS2R19, OR2J3, OR2J3, OR5A1, CD36, TAS2R50, SCNN1D, ORD74, TRPA1, TAS2R60, TAS2R20, GNAT3, ORD74, TAS2R38, GHRL, TAS2R46, OR2J3, TAS1R3, TAS1R2, TRPV1, TAS2R43, HLA-DOA, TAS1R1, and TAS2R16. In some embodiments, the plurality of targets comprises OR10A6, OR10A2, OR10A4, OR10A3, OR6A2, GHRL, TAS2R46, TAS2R43, and TAS2R20. In some embodiments, the plurality of targets comprises OR10A6, GHRL, TAS2R46, TAS2R43, and TAS2R20. In some embodiments, the plurality of targets comprises OR10A2, GHRL, TAS2R46, TAS2R43, and TAS2R20. In some embodiments, the plurality of targets comprises OR10A4, GHRL, TAS2R46, TAS2R43, and TAS2R20. In some embodiments, the plurality of targets comprises OR10A3, GHRL, TAS2R46, TAS2R43, and TAS2R20. In some embodiments, the plurality of targets comprises OR6A2, GHRL, TAS2R46, TAS2R43, and TAS2R20. In some embodiments, the plurality of targets comprises GHRL and TAS2R46. In some embodiments, the plurality of targets comprises GHRL and TAS2R43. In some embodiments, the plurality of targets comprises GHRL and TAS2R20. In some embodiments, the plurality of targets comprises TAS2R46 and TAS2R43. In some embodiments, the plurality of targets comprises TAS2R46 and TAS2R20. In some embodiments, the plurality of targets comprises TAS2R43 and TAS2R20. In some embodiments, the plurality of targets comprises at least 2, 3, 4, 5, 10, 15, 20, 25, 30, 35 or 41 targets selected from Table 1.

In some embodiments, the present invention provides a method for predicting wine preferences of a subject, the method comprising: a) providing a biological sample comprising DNA from the subject; b) genotyping a plurality of SNPs in the sample, wherein the plurality of SNPs comprises one or more SNPs selected from Table 1; c) obtaining taste preference information from the subject; and d) determining the subject's taste preference scores for one or more wines, thereby predicting wine preferences for the subject. In some embodiments, the method further comprises: calculating wine bin scores based on the genotyping from step (b), the information obtained about the subject from step (c), and the taste preference scores for the one or more wines from step (d). In some aspects, the wine bins comprise one or more wine bins selected from the group consisting of a first wine bin for sweet white wines, a second wine bin for crisp and citrusy white wines, a third wine bin for crisp and floral white wines, a fourth wine bin for unoaked full-bodied white wines, a fifth wine bin for oaked full-bodied white wines, a sixth wine bin for light cherry red wines, a seventh wine bin for light chocolaty raspberry red wines, and an eighth wine bin for bold smoky blackberry red wines. In certain aspects, the wine bin scores are used to predict that the subject will prefer wines from a wine bin having a higher wine bin score than a wine bin having a lower wine bin score. The method may be performed prior to serving a wine to the subject, prior to selling a wine to the subject, or prior to recommending a wine to the subject. In one embodiment, the method further comprises creating and storing a user taste profile comprising information about the subject's wine preferences based on the subject's wine bin scores. In other embodiments, the method further comprises providing the subject with at least one sample of wine from the wine bin having the highest wine bin score. Additionally the subject may be provided with one or more wine samples from other wine bins, preferably the top scoring wine bins. For example, the subject may be provided with one or more wine samples from one or more of the wine bins having the top two, three, or four wine bin scores. In some embodiments, the one or more SNPs comprise rs838133, rs1421085, rs3930459, rs2274333, rs111615792, rs72921001, rs7792845, rs10772420, rs846672, rs9295791, rs6591536, rs1761667, rs10772397, rs586965, rs5020278, rs11988795, rs4595035, rs12226920, rs1524600, rs61729907, rs1548803, rs4684677, rs42451, rs35680, rs34911341, rs696217, rs26802, rs2708377, rs28757581, rs35744813, rs4920566, rs3935570, rs2277675, rs71443637, rs9276975, rs34160967, rs307377, rs713598, rs1726866, rs10246939, and rs846664.

In other embodiments, the invention provides a method for providing a wine to a subject, the method comprising the following steps: (a) providing a biological sample comprising DNA from the subject; (b) genotyping the sample to determine which alleles are present at single nucleotide polymorphisms comprising rs838133, rs1421085, rs3930459, rs2274333, rs111615792, rs72921001, rs7792845, rs10772420, rs846672, rs9295791, rs6591536, rs1761667, rs10772397, rs586965, rs5020278, rs11988795, rs4595035, rs12226920, rs1524600, rs61729907, rs1548803, rs4684677, rs42451, rs35680, rs34911341, rs696217, rs26802, rs2708377, rs28757581, rs35744813, rs4920566, rs3935570, rs2277675, rs71443637, rs9276975, rs34160967, rs307377, rs713598, rs1726866, rs10246939, and rs846664; (c) obtaining information about the subject; (d) determining the subject's taste preference scores for one or more wines comprising Riesling, Sauvignon Blanc, Chardonnay, Pinot Noir, Grenache, Zinfandel, Syrah, and Cabernet Sauvignon; and (e) calculating wine bin scores for one or more wine bins based on the genotyping from step (b), the information obtained about the subject from step (c), and the taste preference scores for one or more wines from step (d); and (f) providing the subject with at least one wine from the wine bin that has the subject's highest wine bin score. In some aspects, the information about the subject is selected from the group consisting of the subject's gender; whether the subject prefers sweet wine or oaked wine; taste preferences of the subject for mushrooms, black coffee, savory flavor, grass, peaches, sweet coffee, Swiss cheese, and blackberries; interest in learning about wines; whether or not the subject tries new wines, and whether or not the subject drinks wine frequently. In other aspects, the wine bins comprise a first wine bin for sweet white wines, a second wine bin for crisp and citrusy white wines, a third wine bin for crisp and floral white wines, a fourth wine bin for unoaked full-bodied white wines, a fifth wine bin for oaked full-bodied white wines, a sixth wine bin for light cherry red wines, a seventh wine bin for light chocolaty raspberry red wines, and an eighth wine bin for bold smoky blackberry red wines. The method may be performed prior to serving a wine to the subject, prior to selling a wine to the subject, or prior to recommending a wine to the subject. In one embodiment, the method further comprises creating and storing a user taste profile comprising information about the subject's wine preferences based on the subject's wine bin scores. In other embodiments, the method further comprises providing the subject with at least one sample of wine from the wine bin having the highest wine bin score. Additionally the subject may be provided with one or more wine samples from other wine bins, preferably the top scoring wine bins. For example, the subject may be provided with one or more wine samples from one or more of the wine bins having the top two, three, or four wine bin scores.

In some embodiments, the present invention provides a method of creating a taste profile for a subject, the method comprising: a) obtaining a biological sample comprising nucleic acids from a subject; b) processing the sample to isolate or enrich the sample for the nucleic acids; and c) assaying a plurality of SNPs in the sample, wherein the plurality of SNPs comprises one or more SNPs selected from Table 1, thereby creating a taste profile for the subject.

In some embodiments, the present invention provides a method of creating a taste profile for a subject, the method comprising: a) providing a biological sample comprising DNA from the subject; b) genotyping a plurality of SNPs in the sample, wherein the plurality of SNPs comprises one or more SNPs selected from Table 1; c) obtaining taste preference information from the subject; and d) determining the subject's taste preference scores for one or more wines, thereby predicting wine preferences for the subject. In some embodiments, the method further comprises: calculating wine bin scores based on the genotyping from step (b), the information obtained about the subject from step (c), and the taste preference scores for the one or more wines from step (d). In some aspects, the wine bins comprise one or more wine bins selected from the group consisting of a first wine bin for sweet white wines, a second wine bin for crisp and citrusy white wines, a third wine bin for crisp and floral white wines, a fourth wine bin for unoaked full-bodied white wines, a fifth wine bin for oaked full-bodied white wines, a sixth wine bin for light cherry red wines, a seventh wine bin for light chocolaty raspberry red wines, and an eighth wine bin for bold smoky blackberry red wines. In certain aspects, the wine bin scores are used to predict that the subject will prefer wines from a wine bin having a higher wine bin score than a wine bin having a lower wine bin score. In other embodiments, the method further comprises creating the taste profile for the subject based on steps (a)-(d). In one embodiment, the method further comprises selecting a wine for the subject based on the wine profile. In another embodiment, the method further comprises storing the taste profile.

In another aspect, the invention includes a method of creating a taste profile for a subject, the method comprising: a) providing a biological sample comprising DNA from the subject; b) genotyping the sample to determine which alleles are present at one or more SNPs selected from Table 1; c) obtaining information about the subject; d) determining the subject's taste preference scores for one or more wines comprising Riesling, Sauvignon Blanc, Chardonnay, Pinot Noir, Grenache, Zinfandel, Syrah, and Cabernet Sauvignon; e) calculating wine bin scores for one or more wine bins based on the genotyping from step (b), the information obtained about the subject from step (c), and the taste preference scores for the one or more wines from step (d), wherein the wine bin scores are used to predict that the subject will prefer wines from a wine bin having a higher wine bin score than a wine bin having a lower wine bin score; and f) creating the taste profile for the subject based on steps (a)-(e). In one embodiment, the method further comprises selecting a wine for the subject based on the wine profile. In another embodiment, the method further comprises storing the taste profile. In some embodiments, the one more SNPs comprise rs838133, rs1421085, rs3930459, rs2274333, rs111615792, rs72921001, rs7792845, rs10772420, rs846672, rs9295791, rs6591536, rs1761667, rs10772397, rs586965, rs5020278, rs11988795, rs4595035, rs12226920, rs1524600, rs61729907, rs1548803, rs4684677, rs42451, rs35680, rs34911341, rs696217, rs26802, rs2708377, rs28757581, rs35744813, rs4920566, rs3935570, rs2277675, rs71443637, rs9276975, rs34160967, rs307377, rs713598, rs1726866, rs10246939, and rs846664. In other embodiments, the information about the subject is selected from the group consisting of the subject's gender; whether the subject prefers sweet wine or oaked wine; taste preferences of the subject for mushrooms, black coffee, savory flavor, grass, peaches, sweet coffee, Swiss cheese, and blackberries; subject's interest in learning about wines; whether or not the subject tries new wines, and whether or not the subject drinks wine frequently. In yet other embodiments, the wine bins comprise a first wine bin for sweet white wines, a second wine bin for crisp and citrusy white wines, a third wine bin for crisp and floral white wines, a fourth wine bin for unoaked full-bodied white wines, a fifth wine bin for oaked full-bodied white wines, a sixth wine bin for light cherry red wines, a seventh wine bin for light chocolaty raspberry red wines, and an eighth wine bin for bold smoky blackberry red wines. In some embodiments, the method further comprises selecting a wine for the subject based on the user taste profile. In other embodiments, the method further comprises storing the user taste profile.

In another aspect the invention includes a method of providing a wine to a subject, the method comprising the following steps: a) providing a biological sample comprising DNA from the subject; b) genotyping the sample to determine which alleles are present at single nucleotide polymorphisms comprising rs838133, rs1421085, rs3930459, rs2274333, rs111615792, rs72921001, rs7792845, rs10772420, rs846672, rs9295791, rs6591536, rs1761667, rs10772397, rs586965, rs5020278, rs11988795, rs4595035, rs12226920, rs1524600, rs61729907, rs1548803, rs4684677, rs42451, rs35680, rs34911341, rs696217, rs26802, rs2708377, rs28757581, rs35744813, rs4920566, rs3935570, rs2277675, rs71443637, rs9276975, rs34160967, rs307377, rs713598, rs1726866, rs10246939, and rs846664; c) obtaining information about the subject; d) determining the subject's taste preference scores for one or more wines comprising Riesling, Sauvignon Blanc, Chardonnay, Pinot Noir, Grenache, Zinfandel, Syrah, and Cabernet Sauvignon; and e) calculating wine bin scores for one or more wine bins based on the genotyping from step (b), the information obtained about the subject from step (c), and the taste preference scores for one or more wines from step (d); and f) providing the subject with at least one wine from the wine bin that has the subject's highest wine bin score. In another embodiment, the method further comprises providing the subject with at least one wine from a wine bins that has a positive ranking. In certain embodiments, the method further comprises providing at least one wine to the subject on a periodic basis. In another embodiment, the method further comprises providing the subject with a plurality of wine samples from one or more of the wine bins having positive rankings or preferably the top two, three, or four wine bin scores. In some embodiments, the information about the subject is selected from the group consisting of the subject's gender; whether the subject prefers sweet wine or oaked wine; taste preferences of the subject for mushrooms, black coffee, savory flavor, grass, peaches, sweet coffee, Swiss cheese, and blackberries; interest in learning about wines; whether or not the subject tries new wines, and whether or not the subject drinks wine frequently. In some embodiments, the wine bins comprise a first wine bin for sweet white wines, a second wine bin for crisp and citrusy white wines, a third wine bin for crisp and floral white wines, a fourth wine bin for unoaked full-bodied white wines, a fifth wine bin for oaked full-bodied white wines, a sixth wine bin for light cherry red wines, a seventh wine bin for light chocolaty raspberry red wines, and an eighth wine bin for bold smoky blackberry red wines.

Genotyping of the single nucleotide polymorphisms in Table 1, such as, for example, rs838133, rs1421085, rs3930459, rs2274333, rs111615792, rs72921001, rs7792845, rs10772420, rs846672, rs9295791, rs6591536, rs1761667, rs10772397, rs586965, rs5020278, rs11988795, rs4595035, rs12226920, rs1524600, rs61729907, rs1548803, rs4684677, rs42451, rs35680, rs34911341, rs696217, rs26802, rs2708377, rs28757581, rs35744813, rs4920566, rs3935570, rs2277675, rs71443637, rs9276975, rs34160967, rs307377, rs713598, rs1726866, rs10246939, and rs846664 can be performed using any method known in the art, including, but not limited to, dynamic allele-specific hybridization (DASH), microarray analysis, Tetra-primer ARMS-PCR, a TaqMan 5′-nuclease assay; an Invader assay with Flap endonuclease (FEN), a Serial Invasive Signal Amplification Reaction (SISAR), an oligonucleotide ligase assay, restriction fragment length polymorphism (RFLP), single-strand conformation polymorphism, temperature gradient gel electrophoresis (TGGE), denaturing high performance liquid chromatography (DHPLC), sequencing, and immunoassay.

In another aspect, the invention includes a computer implemented method for predicting wine preferences of a subject, the computer performing steps comprising: a) receiving inputted data comprising: i) genotyping information for the subject regarding which alleles are present at one or more SNPs selected from Table 1, ii) values for the subject's taste preference scores for one or more wines comprising Riesling, Sauvignon Blanc, Chardonnay, Pinot Noir, Grenache, Zinfandel, Syrah, and Cabernet Sauvignon, and iii) information about the subject; b) calculating the subject's wine bin scores for one or more wine bins based on the subject's taste preference scores for one or more wines, wherein the wine bin scores are used to predict that the subject will prefer wines from a wine bin having a higher wine bin score than a wine bin having a lower wine bin score; and c) displaying information regarding predicted wine preferences of the subject. In one embodiment, the computer implemented method further comprises storing a user profile for the subject comprising information regarding the subject's wine preferences. In other embodiments, the information about the subject is selected from the group consisting of the subject's gender; whether the subject prefers sweet wine or oaked wine; taste preferences of the subject for mushrooms, black coffee, savory flavor, grass, peaches, sweet coffee, Swiss cheese, and blackberries; subject's interest in learning about wines; whether or not the subject tries new wines, and whether or not the subject drinks wine frequently. In still other embodiments, the wine bins comprise a first wine bin for sweet white wines, a second wine bin for crisp and citrusy white wines, a third wine bin for crisp and floral white wines, a fourth wine bin for unoaked full-bodied white wines, a fifth wine bin for oaked full-bodied white wines, a sixth wine bin for light cherry red wines, a seventh wine bin for light chocolaty raspberry red wines, and an eighth wine bin for bold smoky blackberry red wines. In other embodiments, the method further comprises creating and storing a user taste profile comprising information about the subject's wine preferences based on the subject's wine bin scores.

In certain embodiments, the computer implemented method further comprises inputting a list of wines from a commercial establishment (e.g., restaurant, winery, bar, or store), and displaying a listing of wines available at the commercial establishment that belong to the wine bin having the highest wine bin score. The list of wines from a commercial establishment may be obtained, for example, by providing an image of a menu from the commercial establishment, and processing the image to obtain the list of wines. Alternatively, the list of wines may be obtained by identifying the commercial establishment, and retrieving an electronic representation of the list of wines from a wine list database. In certain embodiments, the computer implemented method further comprises displaying a listing of wines available at a commercial establishment that belong to the wine bins having the top two, three, or four wine bin scores. In another embodiment, the computer implemented method further comprises displaying a listing of wines available at a commercial establishment that belong to the wine bins having positive rankings.

In another aspect, the invention includes a system for performing the computer implemented method for predicting wine preferences of a subject, as described herein. The system comprises: a) a storage component for storing data, wherein the storage component has instructions for determining the wine preferences of the subject stored therein; b) a computer processor for processing data, wherein the computer processor is coupled to the storage component and configured to execute the instructions stored in the storage component in order to receive data regarding the subject and analyze data according to one or more algorithms; and c) a display component for displaying information regarding the wine preferences of the subject.

In another aspect, the invention includes a kit for creating a user taste profile, the kit comprising at least one agent for determining which alleles are present at one or more SNPs selected from Table 1. In certain embodiments, the one or more SNPs rs838133, rs1421085, rs3930459, rs2274333, rs111615792, rs72921001, rs7792845, rs10772420, rs846672, rs9295791, rs6591536, rs1761667, rs10772397, rs586965, rs5020278, rs11988795, rs4595035, rs12226920, rs1524600, rs61729907, rs1548803, rs4684677, rs42451, rs35680, rs34911341, rs696217, rs26802, rs2708377, rs28757581, rs35744813, rs4920566, rs3935570, rs2277675, rs71443637, rs9276975, rs34160967, rs307377, rs713598, rs1726866, rs10246939, and rs846664. In certain embodiments, the kit comprises a set of allele-specific probes that hybridize to nucleic acids comprising one or more SNPs selected from Table 1. In other embodiments, set of allele-specific probes that hybridize to nucleic acids comprise rs838133, rs1421085, rs3930459, rs2274333, rs111615792, rs72921001, rs7792845, rs10772420, rs846672, rs9295791, rs6591536, rs1761667, rs10772397, rs586965, rs5020278, rs11988795, rs4595035, rs12226920, rs1524600, rs61729907, rs1548803, rs4684677, rs42451, rs35680, rs34911341, rs696217, rs26802, rs2708377, rs28757581, rs35744813, rs4920566, rs3935570, rs2277675, rs71443637, rs9276975, rs34160967, rs307377, rs713598, rs1726866, rs10246939, and rs846664. In another embodiment, the kit comprises a SNP array comprising a set of allele-specific probes that hybridize to nucleic acids comprising one or more SNPs selected from Table 1. In some embodiments the SNPs comprise rs838133, rs1421085, rs3930459, rs2274333, rs111615792, rs72921001, rs7792845, rs10772420, rs846672, rs9295791, rs6591536, rs1761667, rs10772397, rs586965, rs5020278, rs11988795, rs4595035, rs12226920, rs1524600, rs61729907, rs1548803, rs4684677, rs42451, rs35680, rs34911341, rs696217, rs26802, rs2708377, rs28757581, rs35744813, rs4920566, rs3935570, rs2277675, rs71443637, rs9276975, rs34160967, rs307377, rs713598, rs1726866, rs10246939, and rs846664. In a further embodiment, the kit comprises a set of allele-specific primers for determining which alleles are present at one or more SNPs selected from Table 1. In other embodiments, kit comprises a set of allele-specific primers for determining which alleles are present rs838133, rs1421085, rs3930459, rs2274333, rs111615792, rs72921001, rs7792845, rs10772420, rs846672, rs9295791, rs6591536, rs1761667, rs10772397, rs586965, rs5020278, rs11988795, rs4595035, rs12226920, rs1524600, rs61729907, rs1548803, rs4684677, rs42451, rs35680, rs34911341, rs696217, rs26802, rs2708377, rs28757581, rs35744813, rs4920566, rs3935570, rs2277675, rs71443637, rs9276975, rs34160967, rs307377, rs713598, rs1726866, rs10246939, and rs846664. Additionally, the kit may further comprise reagents for performing dynamic allele-specific hybridization (DASH), Tetra-primer ARMS-PCR, a TaqMan 5′-nuclease assay; an Invader assay with Flap endonuclease (FEN), a Serial Invasive Signal Amplification Reaction (SISAR), an oligonucleotide ligase assay, restriction fragment length polymorphism (RFLP), single-strand conformation polymorphism, temperature gradient gel electrophoresis (TGGE), denaturing high performance liquid chromatography (DHPLC), sequencing, or an immunoassay. The kit may further comprise samples of wines including, for example, Riesling, Sauvignon Blanc, Chardonnay, Pinot Noir, Grenache, Zinfandel, Syrah, and Cabernet Sauvignon. Additionally, the kit may further comprise a questionnaire comprising questions regarding the subject's gender; whether the subject prefers sweet wine or oaked wine; taste preferences of the subject for mushrooms, black coffee, savory flavor, grass, peaches, sweet coffee, Swiss cheese, and blackberries; subject's interest in learning about wines; whether or not the subject tries new wines, and whether or not the subject drinks wine frequently. The kit may further comprise information, in electronic or paper form, comprising instructions on how to determine wine preferences of a subject.

In another aspect, the invention includes a set of allele-specific probes and/or primers for detecting which alleles are present at one or more SNPs selected from Table 1. In other embodiments, the invention includes a set of allele-specific probes and/or primers for detecting which alleles are present at rs3930459, rs42451, rs2708377, rs71443637, and rs12226920. In certain embodiments, the set of allele-specific probes and/or primers further comprises one or more allele-specific probes and/or primers for detecting which alleles are present at one or more single nucleotide polymorphisms selected from the group consisting of rs838133, rs1421085, rs3930459, rs2274333, rs111615792, rs72921001, rs7792845, rs10772420, rs846672, rs9295791, rs6591536, rs1761667, rs10772397, rs586965, rs5020278, rs11988795, rs4595035, rs12226920, rs1524600, rs61729907, rs1548803, rs4684677, rs42451, rs35680, rs34911341, rs696217, rs26802, rs2708377, rs28757581, rs35744813, rs4920566, rs3935570, rs2277675, rs71443637, rs9276975, rs34160967, rs307377, rs713598, rs1726866, rs10246939, and rs846664. In another embodiment, the allele-specific probes are provided by a SNP array.

The methods of the invention make possible targeted marketing and distribution of wines likely to please consumers.

These and other embodiments of the subject invention will readily occur to those of skill in the art in view of the disclosure herein.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

FIG. 1 shows hierarchical clustering of wine preferences.

FIG. 2 shows relationships between wine preferences, also gauged using consensus clustering, which allowed the determination of a reduced number of dimensions that could be used to describe wine preference.

FIG. 3A shows hierarchical clustering of taste perception within twelve wines. FIG. 3B shows clustering of wine preference and genetic variants.

FIG. 4 shows the relationship between the model score and the actual wine preference.

FIG. 5 shows a schematic of the method for assigning wines to a subject.

### DETAILED DESCRIPTION OF THE INVENTION

The practice of the present invention will employ, unless otherwise indicated, conventional methods of genetics, biochemistry, molecular biology and recombinant DNA techniques, within the skill of the art. Such techniques are explained fully in the literature. See, e.g., Single Nucleotide Polymorphisms: Methods and Protocols (Methods in Molecular Biology, A.A. Komar ed., Humana Press; 2^{nd} edition, 2009); Genetic Variation: Methods and Protocols (Methods in Molecular Biology, M.R. Barnes and G. Breen eds., Humana Press, 2010); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.). All publications, patents and patent applications cited herein, whether supra or infra, are hereby incorporated by reference in their entireties.

### I. Definitions

In describing the present invention, the following terms will be employed, and are intended to be defined as indicated below.

It must be noted that, as used in this specification and the appended claims, the singular forms “a,” “an” and “the” include plural referents unless the content clearly dictates otherwise. Thus, for example, reference to “a nucleic acid” includes a mixture of two or more such nucleic acids, and the like.

The terms “polymorphism,” “polymorphic nucleotide,” “polymorphic site” or “polymorphic nucleotide position” refer to a position in a nucleic acid that possesses the quality or character of occurring in several different forms. A nucleic acid polymorphism is characterized by two or more “alleles,” or versions of the nucleic acid sequence. Typically, an allele of a polymorphism that is identical to a reference sequence is referred to as a “reference allele” and an allele of a polymorphism that is different from a reference sequence is referred to as an “alternate allele,” or sometimes a “variant allele.” As used herein, the term “major allele” refers to the more frequently occurring allele at a given polymorphic site, and “minor allele” refers to the less frequently occurring allele, as present in the general or study population.

The term “single nucleotide polymorphism” or “SNP” refers to a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). A single nucleotide polymorphism usually arises due to substitution of one nucleotide for another at the polymorphic site. Single nucleotide polymorphisms can also arise from a deletion of a nucleotide or an insertion of a nucleotide relative to a reference allele.

SNPs generally are described as having a minor allele frequency, which can vary between populations, but generally refers to the sequence variation (A, T, G, or C) that is less common than the major allele. The frequency can be obtained from dbSNP or other sources, or may be determined for a certain population using Hardy-Weinberg equilibrium (See for details see Eberle M A, Rieder M J, Kruglyak L, Nickerson D A (2006) Allele Frequency Matching Between SNPs Reveals an Excess of Linkage Disequilibrium in Genic Regions of the Human Genome. PLoS Genet 2(9): e142. doi:10.1371/journal.pgen.0020142; herein incorporated by reference).

Further details of the SNPs described here can be found in the National Center for Biotechnology Information (NCBI) SNP database, available online at ncbi.nlm.nih.gov/projects/SNP, from which the following brief descriptions are taken. Population data on these SNPs may be found at this site. The SNP representations are according to the NCBI SNP database conventions, and show the two alleles between brackets.

The term “rs3930459,” as used herein, refers to the SNP at position 26 (indicated by the [C/T]) of the following sequence in a non-coding region within a cluster of 56 OR genes: AGTCATCAAGATGTAGAGTCATGAA[C/T]TGTACACTTTACATACATTGGATTT (SEQ ID NO:1). This SNP has a global minor allele frequency (MAF)/MinorAlleleCount of C=0.4141/2074, as reported in the NCBI dbSNP.

The term “rs42451,” as used herein, refers to the SNP at position 26 (indicated by the [C/T]) of the following sequence within the GHRL gene: AGCGAGCAGGGGCTGGGGGAGCTTC[C/T]GACGGCTTTATTCCTCGACTGGAGA (SEQ ID NO:2). This SNP has a global minor allele frequency (MAF)/MinorAlleleCount of T=0.2037/1020, as reported in the NCBI dbSNP.

The term “rs2708377,” as used herein, refers to the SNP at position 26 (indicated by the [A/G]) of the following sequence within the TAS2R46 gene: AACAAACCTTTGCAATTTCCCATCT[A/G]TTTTTGCTTTTGGCTCCTGAATTCC (SEQ ID NO:3). This SNP has a global MAF/MinorAlleleCount of C=0.2406/1205, as reported in the NCBI dbSNP.

The term “rs71443637,” as used herein, refers to the SNP at position 26 (indicated by the [C/T]) of the following sequence within the TAS2R43 gene: GCTGGGATCTTGAGATCCTTTACCA[C/T]GGAGCTGCATCTTCTTGAGATGTTT (SEQ ID NO:4). This SNP has a global MAF/MinorAlleleCount of T=0.3782/1894, as reported in the NCBI dbSNP.

The term “rs12226920,” as used herein, refers to the SNP at position 26 (indicated by the [G/T]) of the following sequence within the TAS2R20 gene: TTATATACGTGTGTTTCATCACAAG[G/T]TGACAAACCAAAAAGAACAAAGACC (SEQ ID NO:5). This SNP has a global MAF/MinorAlleleCount of T=0.4231/2119, as reported in the NCBI dbSNP.

As used herein, the term “probe” refers to a polynucleotide that contains a nucleic acid sequence complementary to a nucleic acid sequence present in the target nucleic acid analyte (e.g., at SNP location). The polynucleotide regions of probes may be composed of DNA, and/or RNA, and/or synthetic nucleotide analogs. Probes may be labeled in order to detect the target sequence. Such a label may be present at the 5′ end, at the 3′ end, at both the 5′ and 3′ ends, and/or internally.

An “allele-specific probe” hybridizes to only one of the possible alleles of a SNP under suitably stringent hybridization conditions.

The term “primer” as used herein, refers to an oligonucleotide that hybridizes to the template strand of a nucleic acid and initiates synthesis of a nucleic acid strand complementary to the template strand when placed under conditions in which synthesis of a primer extension product is induced, i.e., in the presence of nucleotides and a polymerization-inducing agent such as a DNA or RNA polymerase and at suitable temperature, pH, metal concentration, and salt concentration. The primer is preferably single-stranded for maximum efficiency in amplification, but may alternatively be double-stranded. If double-stranded, the primer can first be treated to separate its strands before being used to prepare extension products. This denaturation step is typically affected by heat, but may alternatively be carried out using alkali, followed by neutralization. Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA or RNA synthesis. Typically, nucleic acids are amplified using at least one set of oligonucleotide primers comprising at least one forward primer and at least one reverse primer capable of hybridizing to regions of a nucleic acid flanking the portion of the nucleic acid to be amplified.

An “allele-specific primer” matches the sequence exactly of only one of the possible alleles of a SNP, hybridizes at the SNP location, and amplifies only one specific allele if it is present in a nucleic acid amplification reaction.

As used herein, the term “user” or “consumer” may refer to any individual person, group of people, or association that purchases, uses, or consumes goods, such as wine. A user taste profile may include a ranking of wines based on wine bin scores derived from SNP genotyping and querying subjects on personal characteristics and taste preferences, as described herein.

### II. Modes of Carrying Out the Invention

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention is based on the discovery of SNPs that can be used to determine wine taste preferences of individuals. Genetic profiling of an individual to identify the alleles present at these SNPs is used in combination with specific questioning about personal characteristics and taste preferences in creating a taste profile for an individual. The methods of the invention can be used for selecting and providing wines to a consumer that have a high probability of meeting the individual's taste preferences.

Detecting and Genotyping Gene Polymorphisms Associated with Wine Taste Preferences

The SNPs listed in Table 1 can be used as genetic markers for determining wine preferences of an individual. In certain embodiments, rs3930459, rs42451, rs2708377, rs71443637, and rs12226920 are used for determining wine preferences of an individual. These genetic markers can be used in combination with wine sampling of a set of typifying wines (i.e. representing different types, varieties, or styles of wine) and querying the individual regarding certain personal characteristics and taste preferences to create a user profile for an individual comprising information regarding likely taste preferences for different wines.

The SNP rs3930459 is located in a non-coding region within a cluster of the 56 OR genes. The major allele of rs3930459 has a T and the minor allele of rs3930459 has a C at position 26 (indicated by the [C/T]) of the following sequence: AGTCATCAAGATGTAGAGTCATGAA[C/T]TGTACACTTTACATACATTGGATTT (SEQ ID NO:1).

The SNP rs42451 is located within the GHRL gene. The major allele of rs42451 has a C and the minor allele of rs42451 has a Tat position 26 (indicated by the [C/T]) of the following sequence: AGCGAGCAGGGGCTGGGGGAGCTTC[C/T]GACGGCTTTATTCCTCGACTGGAGA (SEQ ID NO:2).

The SNP rs2708377 is located within the TAS2R46 gene. The major allele of rs2708377 has a T and the minor allele of rs2708377 has a C at position 26 (indicated by the [A/G] (allele reported in reverse orientation to the genome)) of the reverse complement of the following sequence: AACAAACCTTTGCAATTTCCCATCT[A/G]TTTTTGCTTTTGGCTCCTGAATTCC (SEQ ID NO:3).

The SNP rs71443637 is located within the TAS2R43 gene. The major allele of rs71443637 has a C and the minor allele of rs71443637 has a T at position 26 (indicated by the [C/T]) of the following sequence: GCTGGGATCTTGAGATCCTTTACCA[C/T]GGAGCTGCATCTTCTTGAGATGTTT (SEQ ID NO:4).

The SNP rs12226920 is located within the TAS2R20 gene. The major allele of rs12226920 has a G and the minor allele of rs12226920 has a T at position 26 (indicated by the [G/T]) of the following sequence: TTATATACGTGTGTTTCATCACAAG[G/T]TGACAAACCAAAAAGAACAAAGACC (SEQ ID NO:5).

SNPs that may be used in the methods and panels of the present invention include rs838133, rs1421085, rs3930459, rs2274333, rs111615792, rs72921001, rs7792845, rs10772420, rs846672, rs9295791, rs6591536, rs1761667, rs10772397, rs586965, rs5020278, rs11988795, rs4595035, rs12226920, rs1524600, rs61729907, rs1548803, rs4684677, rs42451, rs35680, rs34911341, rs696217, rs26802, rs2708377, rs28757581, rs35744813, rs4920566, rs3935570, rs2277675, rs71443637, rs9276975, rs34160967, rs307377, rs713598, rs1726866, rs10246939, and rs846664. Generally, the methods of the present invention comprise: a) obtaining a biological sample comprising nucleic acids from a subject; b) processing the sample to isolate or enrich the sample for the nucleic acids; and c) analyzing the genotype of the biological sample at a plurality of SNPs in a panel of SNPs identified as useful for identifying taste and/or scent preference. In some instances the plurality of SNPs comprises one or more SNPs selected from Table 1. In some instances, the plurality of targets comprises at least about 2, at least about 3, at least about 4, at least about 5, at least about 6, at least about 7, at least about 8, at least about 9, at least about 10, at least about 15, at least about 20, at least about 30, at least about 40, or at least about 41 SNPs from Table 1.

For genetic testing, a biological sample containing nucleic acids is collected from an individual. The biological sample is typically saliva or cells from buccal swabbing, but can be any sample from bodily fluids, tissue or cells that contains genomic DNA or RNA of the individual. In certain embodiments, nucleic acids from the biological sample are isolated, purified, and/or amplified prior to analysis using methods well-known in the art. See, e.g., Green and Sambrook Molecular Cloning: A Laboratory Manual (Cold Spring Harbor Laboratory Press; 4^{th} edition, 2012); and Current Protocols in Molecular Biology (Ausubel ed., John Wiley & Sons, 1995); herein incorporated by reference in their entireties.

SNPs can be detected in a sample by any suitable method known in the art. Detection of a SNP can be direct or indirect. For example, the SNP itself can be detected directly. Alternatively, the SNP can be detected indirectly from cDNAs, amplified RNAs or DNAs, or proteins expressed by the SNP allele. Any method that detects a single base change in a nucleic acid sample can be used. For example, allele-specific probes that specifically hybridize to a nucleic acid containing the polymorphic sequence can be used to detect SNPs. A variety of nucleic acid hybridization formats are known to those skilled in the art. For example, common formats include sandwich assays and competition or displacement assays. Hybridization techniques are generally described in Hames, and Higgins “Nucleic Acid Hybridization, A Practical Approach,” IRL Press (1985); Gall and Pardue, Proc. Natl. Acad. Sci. U.S.A., 63:378-383 (1969); and John et al Nature, 223:582-587 (1969).

Sandwich assays are commercially useful hybridization assays for detecting or isolating nucleic acids. Such assays utilize a “capture” nucleic acid covalently immobilized to a solid support and a labeled “signal” nucleic acid in solution. The clinical sample will provide the target nucleic acid. The “capture” nucleic acid and “signal” nucleic acid probe hybridize with the target nucleic acid to form a “sandwich” hybridization complex.

In one embodiment, the allele-specific probe is a molecular beacon. Molecular beacons are hairpin shaped oligonucleotides with an internally quenched fluorophore. Molecular beacons typically comprise four parts: a loop of about 18-30 nucleotides, which is complementary to the target nucleic acid sequence; a stem formed by two oligonucleotide regions that are complementary to each other, each about 5 to 7 nucleotide residues in length, on either side of the loop; a fluorophore covalently attached to the 5′ end of the molecular beacon, and a quencher covalently attached to the 3′ end of the molecular beacon. When the beacon is in its closed hairpin conformation, the quencher resides in proximity to the fluorophore, which results in quenching of the fluorescent emission from the fluorophore. In the presence of a target nucleic acid having a region that is complementary to the strand in the molecular beacon loop, hybridization occurs resulting in the formation of a duplex between the target nucleic acid and the molecular beacon. Hybridization disrupts intramolecular interactions in the stem of the molecular beacon and causes the fluorophore and the quencher of the molecular beacon to separate resulting in a fluorescent signal from the fluorophore that indicates the presence of the target nucleic acid sequence.

For SNP detection, the molecular beacon is designed to only emit fluorescence when bound to a specific allele of a SNP. When the molecular beacon probe encounters a target sequence with as little as one non-complementary nucleotide, the molecular beacon preferentially stay in its natural hairpin state and no fluorescence is observed because the fluorophore remains quenched. See, e.g., Nguyen et al. (2011) Chemistry 17(46):13052-13058; Sato et al. (2011) Chemistry 17(41):11650-11656; Li et al. (2011) Biosens Bioelectron. 26(5):2317-2322; Guo et al. (2012) Anal. Bioanal. Chem. 402(10):3115-3125; Wang et al. (2009) Angew. Chem. Int. Ed. Engl. 48(5):856-870; and Li et al. (2008) Biochem. Biophys. Res. Commun. 373(4):457-461; herein incorporated by reference in their entireties.

In another embodiment, detection of the SNP sequence is performed using allele-specific amplification. In the case of PCR, amplification primers can be designed to bind to a portion of one of the disclosed genes, and the terminal base at the 3′ end is used to discriminate between the major and minor alleles or mutant and wild-type forms of the genes. If the terminal base matches the major or minor allele, polymerase-dependent three prime extension can proceed. Amplification products can be detected with specific probes. This method for detecting point mutations or polymorphisms is described in detail by Sommer et al. in Mayo Clin. Proc. 64:1361-1372 (1989).

Tetra-primer ARMS-PCR uses two pairs of primers that can amplify two alleles of a SNP in one PCR reaction. Allele-specific primers are used that hybridize at the SNP location, but each matches perfectly to only one of the possible alleles. If a given allele is present in the PCR reaction, the primer pair specific to that allele will amplify that allele, but not the other allele of the SNP. The two primer pairs for the different alleles may be designed such that their PCR products are of significantly different length, which allows them to be distinguished readily by gel electrophoresis. See, e.g., Mufloz et al. (2009) J. Microbiol. Methods. 78(2):245-246 and Chiapparino et al. (2004) Genome. 47(2):414-420; herein incorporated by reference.

SNPs may also be detected by ligase chain reaction (LCR) or ligase detection reaction (LDR). The specificity of the ligation reaction is used to discriminate between the major and minor alleles of the SNP. Two probes are hybridized at the SNP polymorphic site of a nucleic acid of interest, whereby ligation can only occur if the probes are identical to the target sequence. See e.g., Psifidi et al. (2011) PLoS One 6(1):e14560; Asari et al. (2010) Mol. Cell. Probes. 24(6):381-386; Lowe et al. (2010) Anal Chem. 82(13):5810-5814; herein incorporated by reference.

SNPs can also be detected in a biological sample by sequencing and SNP typing. In the former method, one simply carries out whole genome sequencing of a DNA sample, and uses the results to detect the present sequences. Whole genome analysis is used in the field of “personal genomics,” and genetic testing services exist, which provide full genome sequencing using massively parallel sequencing. Massively parallel sequencing is described e.g. in U.S. Pat. No. 5,695,934, entitled “Massively parallel sequencing of sorted polynucleotides,” and US 2010/0113283 A1, entitled “Massively multiplexed sequencing.” Massively parallel sequencing typically involves obtaining DNA representing an entire genome, fragmenting it, and obtaining millions of random short sequences, which are assembled by mapping them to a reference genome sequence.

Commercial services are also available that are capable of genotyping approximately 1 million SNPs for a fixed fee. SNP analysis can be carried out with a variety of methods that do not involve massively parallel random sequencing. For example, a commercially available MassARRAY system can be used. This system uses matrix-assisted laser desorption ionization time-of-flight mass spectrometry (MALDI-TOF MS) coupled with single-base extension PCR for high-throughput multiplex SNP detection. Another commercial SNP system, the Illumina Golden Gate assay, generates SNP specific PCR products that are subsequently hybridized to beads either on a solid matrix or in solution. Three oligonucleotides are synthesized for each SNP: two allele specific oligonucleotides (ASOs) that distinguish the SNP, and a locus specific sequence (LSO) just downstream of the SNP. The ASO and LSO sequences also contain target sequences for a set of universal primers (P1 through P3 in the adjacent figure), while each LSO also contains a particular address sequences (the “illumicode”) complementary to sequences attached to beads.

As another example, a SNP array comprising probes for detecting SNPs can be used. For example, SNP arrays are commercially available from Affymetrix and Illumina, which use multiple sets of short oligonucleotide probes for detecting known SNPs. The design of SNP arrays, such as manufactured by Affymetrix or Illumina, is described further in LaFamboise, “Single nucleotide polymorphism arrays: a decade of biological, computational and technological advances,” Nuc. Acids Res. 37(13):4181-4193 (2009).

Another method useful in SNP analysis is PCR-dynamic allele specific hybridization (DASH), which involves dynamic heating and coincident monitoring of DNA denaturation, as disclosed by Howell et al. (Nat. Biotech. 17:87-88, 1999). A target sequence is amplified (e.g., by PCR) using one biotinylated primer. The biotinylated product strand is bound to a streptavidin-coated microtiter plate well (or other suitable surface), and the non-biotinylated strand is rinsed away with alkali wash solution. An oligonucleotide probe, specific for one allele (e.g., the wild-type allele), is hybridized to the target at low temperature. This probe forms a duplex DNA region that interacts with a double strand-specific intercalating dye. When subsequently excited, the dye emits fluorescence proportional to the amount of double-stranded DNA (probe-target duplex) present. The sample is then steadily heated while fluorescence is continually monitored. A rapid fall in fluorescence indicates the denaturing temperature of the probe-target duplex. Using this technique, a single-base mismatch between the probe and target results in a significant lowering of melting temperature (Tm) that can be readily detected.

A variety of other techniques can be used to detect polymorphisms, including but not limited to, the Invader assay with Flap endonuclease (FEN), the Serial Invasive Signal Amplification Reaction (SISAR), the oligonucleotide ligase assay, restriction fragment length polymorphism (RFLP), single-strand conformation polymorphism, temperature gradient gel electrophoresis (TGGE), and denaturing high performance liquid chromatography (DHPLC). See, for example Molecular Analysis and Genome Discovery (R. Rapley and S. Harbron eds., Wiley 1^{st} edition, 2004); Jones et al. (2009) New Phytol. 183(4):935-966; Kwok et al. (2003) Curr Issues Mol. Biol. 5(2):43-60; Muñoz et al. (2009) J. Microbiol. Methods. 78(2):245-246; Chiapparino et al. (2004) Genome. 47(2):414-420; Olivier (2005) Mutat Res. 573(1-2):103-110; Hsu et al. (2001) Clin. Chem. 47(8):1373-1377; Hall et al. (2000) Proc. Natl. Acad. Sci. U.S.A. 97(15):8272-8277; Li et al. (2011) J. Nanosci. Nanotechnol. 11(2):994-1003; Tang et al. (2009) Hum. Mutat. 30(10):1460-1468; Chuang et al. (2008) Anticancer Res. 28(4A):2001-2007; Chang et al. (2006) BMC Genomics 7:30; Galeano et al. (2009) BMC Genomics 10:629; Larsen et al. (2001) Pharmacogenomics 2(4):387-399; Yu et al. (2006) Curr. Protoc. Hum. Genet. Chapter 7: Unit 7.10; Lilleberg (2003) Curr. Opin. Drug Discov. Devel. 6(2):237-252; and U.S. Pat. Nos. 4,666,828; 4,801,531; 5,110,920; 5,268,267; 5,387,506; 5,691,153; 5,698,339; 5,736,330; 5,834,200; 5,922,542; and 5,998,137 for a description of such methods; herein incorporated by reference in their entireties.

If the polymorphism is located in the coding region of a gene of interest, the SNP can be identified indirectly by detection of the variant protein produced by the SNP. Variant proteins (i.e., containing an amino acid substitution encoded by the SNP) can be detected using antibodies specific for the variant protein. For example, immunoassays that can be used to detect variant proteins produced by SNPs include, but are not limited to, immunohistochemistry (IHC), western blotting, enzyme-linked immunosorbent assay (ELISA), radioimmunoassays (RIA), “sandwich” immunoassays, fluorescent immunoassays, and immunoprecipitation assays, the procedures of which are well known in the art (see, e.g., Schwarz et al. (2010) Clin. Chem. Lab. Med. 48(12):1745-1749; The Immunoassay Handbook (D.G. Wild ed., Elsevier Science; 3^{rd} edition, 2005); Ausubel et al, eds, 1994, Current Protocols in Molecular Biology, Vol. 1 (John Wiley & Sons, Inc., New York); Coligan Current Protocols in Immunology (1991); Harlow & Lane, Antibodies: A Laboratory Manual (1988); Handbook of Experimental Immunology, Vols. I-IV (D.M. Weir and C.C. Blackwell eds., Blackwell Scientific Publications); herein incorporated by reference herein in their entireties).

### Sets of Allele-Specific Probes and Primers

The present invention also provides a probe set for predicting wine taste preferences of a subject comprising a plurality of allele-specific probes for detecting which alleles are present at one or more SNPs in Table 1. In certain embodiments, the invention provides a probe set for predicting wine taste preferences of a subject comprising a plurality of allele-specific probes for detecting which alleles are present at the SNPs: rs3930459, rs42451, rs2708377, rs71443637, and rs12226920.

The probe set may comprise one or more allele-specific polynucleotide probes. An allele-specific probe hybridizes to only one of the possible alleles of a SNP under suitably stringent hybridization conditions. Individual polynucleotide probes comprise a nucleotide sequence derived from the nucleotide sequence of the target SNP sequences or complementary sequences thereof. The nucleotide sequence of the polynucleotide probe is designed such that it corresponds to, or is complementary to the target SNP sequences. The allele-specific polynucleotide probe can specifically hybridize under either stringent or lowered stringency hybridization conditions to a region of the target sequences, to the complement thereof, or to a nucleic acid sequence (such as a cDNA) derived therefrom.

The selection of the allele-specific polynucleotide probe sequences and determination of their uniqueness may be carried out in silico using techniques known in the art, for example, based on a BLASTN search of the polynucleotide sequence in question against gene sequence databases, such as the Human Genome Sequence, UniGene, dbEST or the non-redundant database at NCBI. In one embodiment of the invention, the allele-specific polynucleotide probe is complementary to the polymorphic region of a single SNP allele target DNA or mRNA sequence. Computer programs can also be employed to select allele-specific probe sequences that may not cross hybridize or may not hybridize non-specifically.

The allele-specific polynucleotide probes of the present invention may range in length from about 15 nucleotides to the full length of the coding target or non-coding target. In one embodiment of the invention, the polynucleotide probes are at least about 15 nucleotides in length. In another embodiment, the polynucleotide probes are at least about 20 nucleotides in length. In a further embodiment, the polynucleotide probes are at least about 25 nucleotides in length. In another embodiment, the polynucleotide probes are between about 15 nucleotides and about 500 nucleotides in length. In other embodiments, the polynucleotide probes are between about 15 nucleotides and about 450 nucleotides, about 15 nucleotides and about 400 nucleotides, about 15 nucleotides and about 350 nucleotides, about 15 nucleotides and about 300 nucleotides, about 15 nucleotides and about 250 nucleotides, about 15 nucleotides and about 200 nucleotides in length. In some embodiments, the probes are at least 15 nucleotides in length. In some embodiments, the probes are at least 15 nucleotides in length. In some embodiments, the probes are at least 20 nucleotides, at least 25 nucleotides, at least 50 nucleotides, at least 75 nucleotides, at least 100 nucleotides, at least 125 nucleotides, at least 150 nucleotides, at least 200 nucleotides, at least 225 nucleotides, at least 250 nucleotides, at least 275 nucleotides, at least 300 nucleotides, at least 325 nucleotides, at least 350 nucleotides, at least 375 nucleotides in length.

The allele-specific polynucleotide probes of a probe set can comprise RNA, DNA, RNA or DNA mimetics, or combinations thereof, and can be single-stranded or double-stranded. Thus the polynucleotide probes can be composed of naturally-occurring nucleobases, sugars and covalent internucleoside (backbone) linkages as well as polynucleotide probes having non-naturally-occurring portions which function similarly. Such modified or substituted polynucleotide probes may provide desirable properties such as, for example, enhanced affinity for a target gene and increased stability. The probe set may comprise a coding target and/or a non-coding target. Preferably, the probe set comprises a combination of a coding target and non-coding target.

In another embodiment, the invention provides a set of allele-specific primers for detecting which alleles are present at one or more SNPs selected from Table 1. In certain embodiments, the detected SNPs comprise rs3930459, rs42451, rs2708377, rs71443637, and rs12226920. An allele-specific primer matches the sequence exactly of only one of the possible alleles of a SNP, hybridizes at the SNP location, and amplifies only one specific allele if it is present in a nucleic acid amplification reaction. For use in amplification reactions such as PCR, a pair of primers can be used for detection of a SNP sequence. Each primer is designed to hybridize selectively to a single allele at the site of the SNP under stringent conditions, particularly under conditions of high stringency, as known in the art. The pairs of allele-specific primers are usually chosen so as to generate an amplification product of at least about 50 nucleotides, more usually at least about 100 nucleotides. Algorithms for the selection of primer sequences are generally known, and are available in commercial software packages. These primers may be used in standard quantitative or qualitative PCR-based assays for SNP genotyping of subjects or biological samples from said subjects. Alternatively, these primers may be used in combination with probes, such as molecular beacons in amplifications using real-time PCR.

A label can optionally be attached to or incorporated into an allele-specific probe or primer polynucleotide to allow detection and/or quantitation of a target SNP polynucleotide. The target SNP polynucleotide may be from genomic DNA, expressed RNA, a cDNA copy thereof, or an amplification product derived therefrom, and may be the positive or negative strand, so long as it can be specifically detected in the assay being used. Similarly, an antibody may be labeled that detects a polypeptide expression product of the SNP allele.

In certain multiplex formats, labels used for detecting different SNPs may be distinguishable. The label can be attached directly (e.g., via covalent linkage) or indirectly, e.g., via a bridging molecule or series of molecules (e.g., a molecule or complex that can bind to an assay component, or via members of a binding pair that can be incorporated into assay components, e.g. biotin-avidin or streptavidin). Many labels are commercially available in activated forms which can readily be used for such conjugation (for example through amine acylation), or labels may be attached through known or determinable conjugation schemes, many of which are known in the art.

Labels useful in the invention described herein include any substance which can be detected when bound to or incorporated into the biomolecule of interest. Any effective detection method can be used, including optical, spectroscopic, electrical, piezoelectrical, magnetic, Raman scattering, surface plasmon resonance, colorimetric, calorimetric, etc. A label is typically selected from a chromophore, a lumiphore, a fluorophore, one member of a quenching system, a chromogen, a hapten, an antigen, a magnetic particle, a material exhibiting nonlinear optics, a semiconductor nanocrystal, a metal nanoparticle, an enzyme, an antibody or binding portion or equivalent thereof, an aptamer, and one member of a binding pair, and combinations thereof. Quenching schemes may be used, wherein a quencher and a fluorophore as members of a quenching pair may be used on a probe, such that a change in optical parameters occurs upon binding to the target introduce or quench the signal from the fluorophore. One example of such a system is a molecular beacon. Suitable quencher/fluorophore systems are known in the art. The label may be bound through a variety of intermediate linkages. For example, a polynucleotide may comprise a biotin-binding species, and an optically detectable label may be conjugated to biotin and then bound to the labeled polynucleotide. Similarly, a polynucleotide sensor may comprise an immunological species such as an antibody or fragment, and a secondary antibody containing an optically detectable label may be added.

Chromophores useful in the methods described herein include any substance which can absorb energy and emit light. For multiplexed assays, a plurality of different signaling chromophores can be used with detectably different emission spectra. The chromophore can be a lumophore or a fluorophore. Typical fluorophores include fluorescent dyes, semiconductor nanocrystals, lanthanide chelates, polynucleotide-specific dyes and green fluorescent protein.

The polynucleotides may be provided in a variety of formats, including as solids, in solution, or in an array. The polynucleotides may optionally comprise one or more labels, which may be chemically and/or enzymatically incorporated into the polynucleotide.

In some embodiments, one or more polynucleotides provided herein can be provided on a substrate. The substrate can comprise a wide range of material, either biological, nonbiological, organic, inorganic, or a combination of any of these. For example, the substrate may be a polymerized Langmuir Blodgett film, functionalized glass, Si, Ge, GaAs, GaP, SiO₂, SiN₄, modified silicon, or any one of a wide variety of gels or polymers such as (poly)tetrafluoroethylene, (poly)vinylidenedifluoride, polystyrene, cross-linked polystyrene, polyacrylic, polylactic acid, polyglycolic acid, poly(lactide coglycolide), polyanhydrides, poly(methyl methacrylate), poly(ethylene-co-vinyl acetate), polysiloxanes, polymeric silica, latexes, dextran polymers, epoxies, polycarbonates, or combinations thereof. Conducting polymers and photoconductive materials can be used.

The substrate can take the form of an array, a photodiode, an optoelectronic sensor such as an optoelectronic semiconductor chip or optoelectronic thin-film semiconductor, or a biochip. The location(s) of probe(s) on the substrate can be addressable; this can be done in highly dense formats, and the location(s) can be microaddressable or nanoaddressable.

### SNP Arrays

The present invention contemplates that a set of allele-specific probes may be provided in an array format. In the context of the present invention, an “array” is a spatially or logically organized collection of polynucleotide probes. An array comprising probes specific for a coding target, non-coding target, or a combination thereof may be used. In some embodiments, an array is used which comprises a wide range of allele-specific probes for detecting one or more SNPs selected from Table 1. In other embodiments, the SNPs detected on the array comprise rs3930459, rs42451, rs2708377, rs71443637, and rs12226920, along with appropriate control sequences.

Typically the allele-specific polynucleotide probes are attached to a solid substrate and are ordered so that the location (on the substrate) and the identity of each are known. The allele-specific polynucleotide probes can be attached to one of a variety of solid substrates capable of withstanding the reagents and conditions necessary for use of the array. Examples include, but are not limited to, polymers, such as (poly)tetrafluoroethylene, (poly)vinylidenedifluoride, polystyrene, polycarbonate, polypropylene and polystyrene; ceramic; silicon; silicon dioxide; modified silicon; (fused) silica, quartz or glass; functionalized glass; paper, such as filter paper; diazotized cellulose; nitrocellulose filter; nylon membrane; and polyacrylamide gel pad. Substrates that are transparent to light are useful for arrays that may be used in an assay that involves optical detection.

Examples of array formats include membrane or filter arrays (for example, nitrocellulose, nylon arrays), plate arrays (for example, multiwell, such as a 24-, 96-, 256-, 384-, 864- or 1536-well, microtitre plate arrays), pin arrays, and bead arrays (for example, in a liquid “slurry”). Arrays on substrates such as glass or ceramic slides are often referred to as chip arrays or “chips.” Such arrays are well known in the art.

### Querying Subjects

Genotyping can be combined with personal information obtained by querying an individual regarding certain personal characteristics and taste preferences to create a user profile comprising information regarding likely taste preferences for different wines.

Subjects are provided with a survey (i.e., questionnaire) comprising questions regarding the subject's gender; whether the subject prefers sweet wine or oaked wine; taste preferences of the subject for mushrooms, black coffee, savory flavor, grass, peaches, sweet coffee, Swiss cheese, and blackberries; subject's interest in learning about wines; whether or not the subject tries new wines, and whether or not the subject drinks wine frequently.

The survey questions may be provided in various formats, for example, orally (e.g., interviewing the subject), in writing (e.g., paper or electronic form), or through a website with interactive tools that guide the subject through the screening process.

### Wine Sampling

In addition, subjects whose wine preferences are to be determined may be offered samples of typifying wines representing different types, varieties, or styles of wine and questioned regarding their preferences among these wines. Typifying wines are chosen to represent one ore more wine categories, including, but not limited to, 1) sweet white wines, 2) crisp and citrusy white wines, 3) crisp and floral white wines, 4) unoaked full-bodied white wines, 5) light, cherry red wines, 6) light chocolaty raspberry red wines, 7) bold smoky blackberry red wines, and 8) bold smoky blackberry red wines.

In some embodiments, the subject is offered one or more wine for each wine category to sample and questioned regarding taste preferences. For each of the typifying wines, taste preferences are recorded, encoded as −1, representing “dislike”, 0, representing “no preference”, and +1, representing “like” the wine.

In one embodiment, samples of a plurality of wines are provided to a subject, including Radius Riesling 2013 (sweet white wine), Kim Crawford Sauvignon Blanc (crisp and citrusy white wine), Cliff Lede Sauvignon Blanc (crisp and floral white wine), Mer Soleil Chardonnay (unoaked full-bodied white wine), Sbragia Chardonnay (oaked full-bodied white wine), Rombauer Chardonnay (oaked full-bodied white wine), Thumbprint Pinot Noir (light, cherry red wine), Siduri Pinot Noir (light, cherry red wine), Grenache (light chocolaty raspberry red wine), Zinfandel (light chocolaty raspberry red wine), Syrah (bold smoky blackberry red wine), and Cabernet Sauvignon (bold smoky blackberry red wine), though as well be apparent, other wines typifying a wine category can be substituted.

### Generating a User Profile

A user profile can be created based on SNP genotyping and analyzing responses to the survey on personal characteristics and taste preferences as described above. In addition, information on wine preferences of a subject from sampling of typifying wines may also be included. The user profile may be provided in a machine (e.g., a computer) readable format and/or in a hard (paper) copy.

In one embodiment, the user profile comprises information regarding a) the subject's genotype at one or more SNPs selected from Table 1; b) information from a survey about the subject; c) the subject's taste preference wine scores for one or more wines comprising Riesling, Sauvignon Blanc, Chardonnay, Pinot Noir, Grenache, Zinfandel, Syrah, and Cabernet Sauvignon; and d) wine bin scores for one or more wine bins based on the genotyping from step (b), the information obtained about the subject from step (c), and the taste preference scores for the one or more wines from step (d), wherein the wine bin scores are used to predict that the subject will prefer wines from a wine bin having a higher wine bin score than a wine bin having a lower wine bin score. In some embodiments, the one or more SNPs comprise rs3930459, rs42451, rs2708377, rs71443637, and rs12226920. In other embodiments, the information about the subject is selected from the group consisting of the subject's gender; whether the subject prefers sweet wine or oaked wine; taste preferences of the subject for mushrooms, black coffee, savory flavor, grass, peaches, sweet coffee, Swiss cheese, and blackberries; subject's interest in learning about wines; whether or not the subject tries new wines, and whether or not the subject drinks wine frequently. In still other embodiments, the wine bins comprise a first wine bin for sweet white wines, a second wine bin for crisp and citrusy white wines, a third wine bin for crisp and floral white wines, a fourth wine bin for unoaked full-bodied white wines, a fifth wine bin for oaked full-bodied white wines, a sixth wine bin for light cherry red wines, a seventh wine bin for light chocolaty raspberry red wines, and an eighth wine bin for bold smoky blackberry red wines.

The relationship between wine preferences and SNP genotyping and survey responses can be evaluated, for example, using hierarchical and k-means clustering, consensus clustering, or ordered logistic regression to provide a wine score for each of the typifying wines (see Example 1). The wine scores of the typifying wines are then used to rank the wine categories (i.e., wine bins) to which the typifying wines belong (see Example 1). Rankings can be represented by a wine bin score, wherein increasing wine bin scores are correlated with increasing preference for a category of wine (i.e., a particular wine bin).

Thus, subjects are given a “wine score” for each typifying wine indicating the degree of preference among the typifying wines, and then given a “wine bin score” for each wine bin in the set of wine bins corresponding to the broad categories of wine to which the typifying wines belong. The wine bin scores are ranked, such that wine bins that are likely to be preferred have higher scores than wine bins that are likely to be less preferred or disliked.

A user taste profile generated in this manner may be used to select wines for an individual. Wines are selected by ranking the wine bins based on their scores, and offering the individual at least one wine from the highest ranked wine bin. If other wine bins have positive rankings, that is, the individual has a higher than average preference for wines from those wine bins, then another wine may be selected from another wine bin having a positive ranking. The subject may be provided with one or more wine samples from other wine bins, preferably the top scoring wine bins. For example, a subject may be provided with one or more wine samples from one or more of the wine bins having the top two, three, or four wine bin scores. Wine bins having negative rankings, indicating that the individual is unlikely to like the wine, are not offered.

In certain embodiments, at least one red wine and at least one white are selected from the highest ranking bins for wines of each color. If the lowest ranked bin of the same wine color (i.e. red or white) as the highest ranked bin is greater than the highest ranked bin of the opposite color, then it may be preferable to only select wines of the preferred color (i.e., red or white), especially if an individual has negative rankings for all wine bins for wines of a particular color.

In certain embodiments, at least one wine is provided to a subject based on the user taste profile on a periodic basis. For example, wines with positive rankings may be selected and shipped periodically as part of a wine club.

### System and Computerized Methods for Creating a Taste Profile

In a further aspect, the invention includes a computer implemented method for creating a taste profile that predicts wine preferences of a subject. The computer performs steps comprising: a) receiving inputted data comprising: i) genotyping information for the subject regarding which alleles are present at one or more SNPs selected from Table 1, ii) values for the subject's taste preference scores for a plurality of typifying wines, and iii) information about the subject; b) calculating the subject's wine bin scores for one or more wine bins based on the subject's taste preference scores for one or more wines, wherein the wine bin scores are used to predict that the subject will prefer wines from a wine bin having a higher wine bin score than a wine bin having a lower wine bin score; and c) displaying information regarding predicted wine preferences of the subject. In one embodiment, the inputted data comprises values for the subject's taste preference scores for one or more wines comprising Riesling, Sauvignon Blanc, Chardonnay, Pinot Noir, Grenache, Zinfandel, Syrah, and Cabernet Sauvignon. In another embodiment, the computer implemented method further comprises storing a user profile for the subject comprising information regarding the subject's wine preferences. In some embodiments, the information about the subject is selected from the group consisting of the subject's gender; whether the subject prefers sweet wine or oaked wine; taste preferences of the subject for mushrooms, black coffee, savory flavor, grass, peaches, sweet coffee, Swiss cheese, and blackberries; subject's interest in learning about wines; whether or not the subject tries new wines, and whether or not the subject drinks wine frequently. In some embodiments, the wine bins comprise a first wine bin for sweet white wines, a second wine bin for crisp and citrusy white wines, a third wine bin for crisp and floral white wines, a fourth wine bin for unoaked full-bodied white wines, a fifth wine bin for oaked full-bodied white wines, a sixth wine bin for light cherry red wines, a seventh wine bin for light chocolaty raspberry red wines, and an eighth wine bin for bold smoky blackberry red wines.

In certain embodiments, the computer implemented method further comprises inputting a list of wines from a commercial establishment (e.g., restaurant, bar, winery, or store), and displaying a listing of wines available at the commercial establishment that belong to the wine bin having the highest wine bin score. The list of wines from a commercial establishment may be obtained, for example, by providing an image of a menu from the commercial establishment, and processing the image to obtain the list of wines. Alternatively, the list of wines may be obtained by identifying the commercial establishment, and retrieving an electronic representation of the list of wines from a wine list database. In certain embodiments, the computer implemented method further comprises displaying a listing of wines available at a commercial establishment that having positive rankings based on their wine bin scores. The wines from different wine bins may be displayed in the order of their ranking and the display may be color coded to differentiate wines from different wine bins or wines of a different color (red wine versus white wines). The display may be adjustable to allow control over how wines are listed. In certain embodiments, the display can be adjusted to list only certain wines with positive rankings that are available at a commercial establishment, such as only wines that belong to the wine bin having the top wine bin score, or wines that belong to wine bins having the top two, three, or four wine bin scores, or only white wines, or only red wines, or any other desired listing. In some embodiments, the one or more SNPs comprise rs3930459, rs42451, rs2708377, rs71443637, and rs12226920.

In a further aspect, the invention includes a system for performing the computer implemented method, as described. The system may include a computer containing a processor, a storage component (i.e., memory), a display component, and other components typically present in general purpose computers. The storage component stores information accessible by the processor, including instructions that may be executed by the processor and data that may be retrieved, manipulated or stored by the processor.

The storage component includes instructions for creating a taste profile that predicts wine preferences of a subject. For example, the storage component includes instructions for calculating taste preference scores for typifying wines and wine bin scores for ranking wines according to predicted preferences of a subject, as described herein (e.g., see Examples). The computer processor is coupled to the storage component and configured to execute the instructions stored in the storage component in order to receive data regarding the subject and analyze data according to one or more algorithms. The display component displays information regarding the predicted wine preferences of the subject.

The storage component may be of any type capable of storing information accessible by the processor, such as a hard-drive, memory card, ROM, RAM, DVD, CD-ROM, USB Flash drive, write-capable, and read-only memories. The processor may be any well-known processor, such as processors from Intel Corporation. Alternatively, the processor may be a dedicated controller such as an ASIC.

The instructions may be any set of instructions to be executed directly (such as machine code) or indirectly (such as scripts) by the processor. In that regard, the terms “instructions,” “steps” and “programs” may be used interchangeably herein. The instructions may be stored in object code form for direct processing by the processor, or in any other computer language including scripts or collections of independent source code modules that are interpreted on demand or compiled in advance.

Data may be retrieved, stored or modified by the processor in accordance with the instructions. For instance, although the diagnostic system is not limited by any particular data structure, the data may be stored in computer registers, in a relational database as a table having a plurality of different fields and records, XML documents, or flat files. The data may also be formatted in any computer-readable format such as, but not limited to, binary values, ASCII or Unicode. Moreover, the data may comprise any information sufficient to identify the relevant information, such as numbers, descriptive text, proprietary codes, pointers, references to data stored in other memories (including other network locations) or information which is used by a function to calculate the relevant data.

In certain embodiments, the processor and storage component may comprise multiple processors and storage components that may or may not be stored within the same physical housing. For example, some of the instructions and data may be stored on a removable DVD and others within a read-only computer chip. Some or all of the instructions and data may be stored in a location physically remote from, yet still accessible by, the processor. Similarly, the processor may actually comprise a collection of processors which may or may not operate in parallel.

In one aspect, the computer is a server communicating with one or more client computers. Each client computer may be configured similarly to the server, with a processor, storage component and instructions. Each client computer may be a personal computer, intended for use by a person, having all the internal components normally found in a personal computer such as a central processing unit (CPU), display (for example, a monitor displaying information processed by the processor), DVD, hard-drive, user input device (for example, a mouse, keyboard, touch-screen or microphone), speakers, modem and/or network interface device (telephone, cable or otherwise) and all of the components used for connecting these elements to one another and permitting them to communicate (directly or indirectly) with one another. Moreover, computers in accordance with the systems and methods described herein may comprise any device capable of processing instructions and transmitting data to and from humans and other computers including network computers lacking local storage capability.

Although the client computers may comprise a full-sized personal computer, many aspects of the system and method are particularly advantageous when used in connection with mobile devices capable of wirelessly exchanging data with a server over a network such as the Internet. For example, client computer may be a wireless-enabled PDA such as a Blackberry phone, Apple iPhone, Android phone, or other Internet-capable cellular phone. In such regard, the user may input information using a small keyboard, a keypad, a touch screen, or any other means of user input. The computer may have an antenna for receiving a wireless signal.

The server and client computers are capable of direct and indirect communication, such as over a network. It should be appreciated that a typical system can include a large number of connected computers, with each different computer being at a different node of the network. The network, and intervening nodes, may comprise various combinations of devices and communication protocols including the Internet, World Wide Web, intranets, virtual private networks, wide area networks, local networks, cell phone networks, private networks using communication protocols proprietary to one or more companies, Ethernet, WiFi and HTTP. Such communication may be facilitated by any device capable of transmitting data to and from other computers, such as modems (e.g., dial-up or cable), networks and wireless interfaces. The server may be a web server.

Although certain advantages are obtained when information is transmitted or received as noted above, other aspects of the system and method are not limited to any particular manner of transmission of information. For example, in some aspects, information may be sent via a medium such as a disk, tape, flash drive, memory card, DVD, or CD-ROM. In other aspects, the information may be transmitted in a non-electronic format and manually entered into the system. Yet further, although some functions are indicated as taking place on a server and others on a client, various aspects of the system and method may be implemented by a single computer having a single processor.

### Kits

Kits for performing the desired method(s) are also provided, and comprise a container or housing for holding the components of the kit, one or more vessels containing one or more nucleic acid(s), and optionally one or more vessels containing one or more reagents. Any composition described herein may be included in the kit, including those reagents useful for performing the methods described, including reagents for SNP genotyping, such as one or more allele specific-probes, primers or primer pairs, enzymes (including polymerases and ligases), intercalating dyes, labeled probes, and labels that can be incorporated into amplification products.

In some embodiments, the kit comprises allele-specific probes and/or primers or primer pairs for determining which alleles are present at one or more SNPs selected from Table 1. In certain embodiments, the SNPs comprise rs3930459, rs42451, rs2708377, rs71443637, rs12226920. The kit may include allele-specific primers or pairs of primers suitable for selectively amplifying the target sequences of a SNP allele. The kit may comprise at least 2, 3, 4, 5, 6, 7, 8, 9, 10, or more allele-specific primers or pairs of primers suitable for selectively amplifying one or more target SNP alleles.

In some embodiments, the primers or primer pairs of the kit, when used in an amplification reaction, specifically amplify a non-coding target, coding target, exonic, or non-exonic target described herein, a nucleic acid sequence corresponding to a SNP allele selected from Table 1, an RNA form thereof, or a complement to either thereof. The kit may include a plurality of such primers or primer pairs which can specifically amplify a corresponding plurality of different target sequence, including a non-coding target, coding target, exonic, or non-exonic transcript described herein, a nucleic acid sequence corresponding to a target selected from Table 1, RNA forms thereof, or complements thereto.

The reagents may independently be in liquid or solid form. The reagents may be provided in mixtures. Control samples and/or nucleic acids may optionally be provided in the kit.

The nucleic acids may be provided in an array format, and thus a SNP array or microarray may be included in the kit. For example, the kit may include a SNP array comprising a set of allele-specific probes for detecting which alleles are present at one or more SNPs selected from Table 1. In certain embodiments, the SNPs comprise rs3930459, rs42451, rs2708377, rs71443637, and rs12226920.

Instructions for using the kit to perform one or more methods of the invention can be provided with the container, and can be provided in any fixed medium. The instructions may be located inside or outside the container or housing, and/or may be printed on the interior or exterior of any surface thereof. A kit may be in multiplex form for concurrently detecting and/or quantitating one or more different target polynucleotides representing the expressed target sequences.

### Data Analysis

In some embodiments, one or more pattern recognition methods can be used in analyzing SNP genotyping data and information regarding a subject's personal characteristics and taste preferences, such as determined from querying the subject and/or wine sampling as described herein. Developing models that predict individual wine preferences may comprise the use of a machine learning algorithm. The machine learning algorithm may comprise a supervised learning algorithm. Examples of supervised learning algorithms may include Average One-Dependence Estimators (AODE), Artificial neural network (e.g., Backpropagation), Bayesian statistics (e.g., Naive Bayes classifier, Bayesian network, Bayesian knowledge base), Case-based reasoning, Decision trees, Inductive logic programming, Gaussian process regression, Group method of data handling (GMDH), Learning Automata, Learning Vector Quantization, Minimum message length (decision trees, decision graphs, etc.), Lazy learning, Instance-based learning Nearest Neighbor Algorithm, Analogical modeling, Probably approximately correct learning (PAC) learning, Ripple down rules, a knowledge acquisition methodology, Symbolic machine learning algorithms, Subsymbolic machine learning algorithms, Support vector machines, Random Forests, Ensembles of classifiers, Bootstrap aggregating (bagging), and Boosting. Supervised learning may comprise ordinal classification such as regression analysis and Information fuzzy networks (IFN). Alternatively, supervised learning methods may comprise statistical classification, such as AODE, Linear classifiers (e.g., Fisher's linear discriminant, Logistic regression, Naive Bayes classifier, Perceptron, and Support vector machine), quadratic classifiers, k-nearest neighbor, Boosting, Decision trees (e.g., C4.5, Random forests), Bayesian networks, and Hidden Markov models.

The machine learning algorithms may also comprise an unsupervised learning algorithm. Examples of unsupervised learning algorithms may include artificial neural network, Data clustering, Expectation-maximization algorithm, Self-organizing map, Radial basis function network, Vector Quantization, Generative topographic map, Information bottleneck method, and IBSEAD. Unsupervised learning may also comprise association rule learning algorithms such as Apriori algorithm, Eclat algorithm and FP-growth algorithm. Hierarchical clustering, such as Single-linkage clustering and Conceptual clustering, may also be used. Alternatively, unsupervised learning may comprise partitional clustering such as K-means algorithm and Fuzzy clustering.

In some instances, the machine learning algorithms comprise a reinforcement learning algorithm. Examples of reinforcement learning algorithms include, but are not limited to, temporal difference learning, Q-learning and Learning Automata. Alternatively, the machine learning algorithm may comprise Data Pre-processing.

Preferably, the machine learning algorithms may include, but are not limited to, Average One-Dependence Estimators (AODE), Fisher's linear discriminant, Logistic regression, Perceptron, Multilayer Perceptron, Artificial Neural Networks, Support vector machines, Quadratic classifiers, Boosting, Decision trees, C4.5, Bayesian networks, Hidden Markov models, High-Dimensional Discriminant Analysis, and Gaussian Mixture Models. The machine learning algorithm may comprise support vector machines, Naive Bayes classifier, k-nearest neighbor, high-dimensional discriminant analysis, or Gaussian mixture models. In some instances, the machine learning algorithm comprises Random Forests.

### III. Experimental

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### EXAMPLES

### Example 1: Genetic Profiling to Predict Individual Wine Taste Preferences and Wine Selection Based on Genetic Profiles

Genetic profiling to predict individual wine taste preferences and selection of wine based on genetic profiles was performed as follows. Adult participants were surveyed concerning their taste preferences, eating and drinking habits, and relevant demographic questions. Additionally, they were offered twelve wines encompassing a number of grape varietals, and questioned on their preferences among these wines and their ability to detect defined flavors in them. A total of 578 questions were asked. The surveys were conducted prior to and at nine different wine tasting events, with 541 people surveyed in total. SurveyMonkey.com was used to digitally collect survey responses from participants. Informed consent for these results to be used for research purposes was obtained.

Subsequently forty-one (41) single nucleotide variants (SNPs) were assayed on all survey participants (Table 1). Variants were chosen by literature review to identify a variety of alleles with likely association with differences in the ability to perceive certain tastes. Saliva was collected by survey participants using a kit supplied by DNA Genotek (Ottawa, Canada) and genomic DNA isolated using the NA Genotek PrepIT*L2P saliva protocol as per manufacturers recommendation. Genotyping was performed by the HudsonAlpha Institute for Biotechnology (Huntsville, Ala.). Briefly, genomic DNA quantity was measured using Invitrogen's PicoGreen assay. Taqman custom and prepared SNP assays were obtained from Applied Biosystems/Thermo. Assays were provided as a probe/primer mix, lyophilized in a 384-well plate. Assays were plated by Thermo in duplicate, with four sets of assays loaded per plate. Genomic DNA samples were mixed with an equivalent amount of 2× master mix in a sterile reservoir, and the mixture was then aliquoted with a Biomek FxP robot to the 384-well plates. The final reaction volume of each well was 5 μl. PCR reactions were run using manufacturer recommended conditions on an ABI thermal cycler (40 cycles). Completed reactions were analyzed using the Applied Biosystems QuantStudio 12K flex system.

TABLE 1
Genetic Variants (SNPs) for Taste Preference

| | | | Major | Minor | | Reference (include | | | Association with |
|---|---|---|---|---|---|---|---|---|---|
| | | | allele | allele | PubMed | first author, title, | | Primary | primary |
| rsId | Chr | Pos | dbSNP | dbSNP | ID | journal, & date) | Gene | phenotype | phenotype |
| rs838133 | 19 | 49259529 | G | A | NA | 23andMe White | FGF21 intron | Sweet: | Higher odds of |
| | | | | | | Paper 23-08, | | preference | preferring sweet- |
| | | | | | | Genetic associations | | | tasting foods over |
| | | | | | | with traits in | | | salty/savory |
| | | | | | | 23andMe customers | | | |
| rs1421085 | 16 | 53800954 | T | C | NA | 23andMe White | FTO intron | Sweet: | Higher odds of |
| | | | | | | Paper 23-08, | | preference | preferring sweet- |
| | | | | | | Genetic associations | | | tasting foods over |
| | | | | | | with traits in | | | salty/savory |
| | | | | | | 23andMe customers | | | |
| rs3930459 | 11 | 7953958 | T | C | NA | 23andMe White | non-coding | Bitter: | Higher odds of |
| | | | | | | Paper 23-08, | regions within | Cilantro | disliking cilantro |
| | | | | | | Genetic associations | cluster of 56 | preference | |
| | | | | | | with traits in | OR genes, | | |
| | | | | | | 23andMe customers | within 10 kb | | |
| | | | | | | | OR10A2, | | |
| | | | | | | | OR10A4, | | |
| | | | | | | | OR10A6, and | | |
| | | | | | | | OR10A3 and | | |
| | | | | | | | within 100 kb | | |
| | | | | | | | of OR6A2 | | |
| | | | | | | | (Xsm 11 OR | | |
| | | | | | | | cluster 1) | | |
| rs2274333 | 1 | 9017204 | A | G | 21712049 | Calo, C., Padiglia, | CA6 (gustin) | Bitter: | Higher bitter taste |
| | | | | | | A., Zonza, A., | | perception | perception, Gustin |
| | | | | | | Corrias, L., Contu, | | | modulates |
| | | | | | | P., Tepper, B. J., et | | | sensitivity to PROP |
| | | | | | | al. (2011). | | | |
| | | | | | | Polymorphisms in | | | |
| | | | | | | TAS2R38 and the | | | |
| | | | | | | taste bud trophic | | | |
| | | | | | | factor, gustin gene | | | |
| | | | | | | cooperate | | | |
| | | | | | | in modulating PROP | | | |
| | | | | | | taste phenotype. | | | |
| | | | | | | Physiology & | | | |
| | | | | | | Behavior, 104(5), | | | |
| | | | | | | 1065-1071. | | | |
| rs111615792 | 1 | 1267651 | G | A | 19587085 | Chen Q Y, Alarcon S, | TAS1R3 | Umami: | Increased umami |
| | | | | | | Tharp A, Ahmed | | perception | taste perception |
| | | | | | | O M, Estrella N L, | | | (doubling of umami |
| | | | | | | Greene T A, Rucker | | | ratings of 200 |
| | | | | | | J, Breslin P A. 2009. | | | mmol MPG/L) |
| | | | | | | Perceptual variation | | | |
| | | | | | | in umami taste and | | | |
| | | | | | | polymorphismsin | | | |
| | | | | | | TAS1R taste | | | |
| | | | | | | receptor genes. Am | | | |
| | | | | | | J Clin Nutr. | | | |
| | | | | | | 90: 770S-779S. | | | |
| rs72921001 | 11 | 6889648 | C | A | NA | Eriksson N, et al. | w/in 100 kb of | Bitter: | soapy taste |
| | | | | | | 2012. A genetic | OR6A2 | Cilantro | detection |
| | | | | | | variant near olfactory | | preference | |
| | | | | | | receptor genes | | (soapy | |
| | | | | | | influences cilantro | | taste) | |
| | | | | | | preference. Flavour | | | |
| | | | | | | 1: 22. | | | |
| rs7792845 | 7 | 80151369 | C | T | 20660057 | Fushan, A. A., | GNAT3 | Sweet: | Increased sucrose |
| | | | | | | Simons, C. T., Slack, | (gustducin) | sucrose | discrimination |
| | | | | | | J. P., & Drayna, D. | | sensitivity | |
| | | | | | | (2010). Association | | | |
| | | | | | | between | | | |
| | | | | | | common variation in | | | |
| | | | | | | genes encoding | | | |
| | | | | | | sweet taste signaling | | | |
| | | | | | | components and | | | |
| | | | | | | human sucrose | | | |
| | | | | | | perception. | | | |
| | | | | | | Chemical Senses, | | | |
| | | | | | | 35(7), 579-592. | | | |
| rs10772420 | 12 | 11174276 | G | A | 21163912 | Hayes, et al. 2011. | TAS2R19 | Bitter: | Less bitterness |
| | | | | | | Allelic variation in | | Grapefruit | perception and |
| | | | | | | TAS2R bitter | | juice | higher liking of |
| | | | | | | receptor genes | | | grapefruit juice |
| | | | | | | associates with | | | |
| | | | | | | variation in | | | |
| | | | | | | sensations from and | | | |
| | | | | | | ingestive behaviors | | | |
| | | | | | | toward common | | | |
| | | | | | | bitter beverages in | | | |
| | | | | | | adults. Chem | | | |
| | | | | | | Senses 36: 311. | | | |
| rs846672 | 7 | 122630180 | C | A | 21163912 | Hayes, et al. 2011. | TAS2R16 | Alcohol | AA consumed |
| | | | | | | Allelic variation in | | trends: | alcohol twice as |
| | | | | | | TAS2R bitter | | consumption | frequently as |
| | | | | | | receptor genes | | | heterozygotes or |
| | | | | | | associates with | | | major allele |
| | | | | | | variation in | | | homozygotes, AA |
| | | | | | | sensations from and | | | also consumed |
| | | | | | | ingestive behaviors | | | larger amounts. |
| | | | | | | toward common | | | |
| | | | | | | bitter beverages in | | | |
| | | | | | | adults. Chem | | | |
| | | | | | | Senses 36: 311. | | | |
| rs9295791 | 6 | 29096033 | A | G | NA | Jaeger, et al. 2010. | OR2J3 (near) | Odor: cis-3-hexen-1-ol | |
| | | | | | | A preliminary | | (grassy odor) detection | |
| | | | | | | investigation into a | | | |
| | | | | | | genetic | | | |
| | | | | | | basis for cis-3- | | | |
| | | | | | | hexen-1-ol odour | | | |
| | | | | | | perception: a | | | |
| | | | | | | genome wide | | | |
| | | | | | | assocation | | | |
| | | | | | | approach. Food | | | |
| | | | | | | Qual Pref 21: 121 | | | |
| rs6591536 | 11 | 59211188 | A | G | 23910657 | Jaeger, et al. 2013. | OR5A1 | Odor: b- | greater sensitivity |
| | | | | | | A Mendelian trait for | | ionone | to |
| | | | | | | olfactory sensitivity | | sensitivity | b-ionone |
| | | | | | | affects odor | | | |
| | | | | | | experience and food | | | |
| | | | | | | selection. Curr Biol | | | |
| | | | | | | 23: 1601. | | | |
| rs1761667 | 7 | 80244939 | G | A | 22240721 | Keller, et al. 2012. | CD36 | Other: Fat | Salad dressings |
| | | | | | | Common variants in | | perception | tasted creamier |
| | | | | | | the CD36 | | | |
| | | | | | | gene are associated | | | |
| | | | | | | with oral fat | | | |
| | | | | | | perception, fat | | | |
| | | | | | | preferences, and | | | |
| | | | | | | obesity | | | |
| | | | | | | in African Americans | | | |
| rs10772397 | 12 | 11138683 | T | C | 22977065 | Knaapila, et al. | TAS2R50 | Bitter: Cilantro pleasantness | |
| | | | | | | 2012. Genetic | | | |
| | | | | | | analysis of | | | |
| | | | | | | chemosensory traits | | | |
| | | | | | | in human twins. | | | |
| | | | | | | Chem Senses | | | |
| | | | | | | 37: 869. | | | |
| rs586965 | 1 | 1226348 | G | C | 22977065 | Knaapila, et al. | SCNN1D | Alcohol trends: Ethanol burn | |
| | | | | | | 2012. Genetic | (salt receptor | | |
| | | | | | | analysis of | gene) | | |
| | | | | | | chemosensory traits | | | |
| | | | | | | in human twins. | | | |
| | | | | | | Chem Senses | | | |
| | | | | | | 37: 869. | | | |
| rs5020278 | 19 | 9325116 | G | A | 22977065 | Knaapila, et al. | ORD74 | Alcohol | Ethanol burn |
| | | | | | | 2012. Genetic | | trends: | |
| | | | | | | analysis of | | Ethanol | |
| | | | | | | chemosensory traits | | burn | |
| | | | | | | in human twins. | | | |
| | | | | | | Chem Senses | | | |
| | | | | | | 37: 869. | | | |
| rs11988795 | 8 | 72949601 | C | T | 22977065 | Knaapila, et al. | TRPA1 | Bitter: Cilantro pleasantness | |
| | | | | | | 2012. Genetic | | | |
| | | | | | | analysis of | | | |
| | | | | | | chemosensory traits | | | |
| | | | | | | in human twins. | | | |
| | | | | | | Chem Senses | | | |
| | | | | | | 37: 869. | | | |
| rs4595035 | 7 | 143141475 | C | T | 22977065 | Knaapila, et al. | TAS2R60 | Bitter: Basil pleasantness | |
| | | | | | | 2012. Genetic | | | |
| | | | | | | analysis of | | | |
| | | | | | | chemosensory traits | | | |
| | | | | | | in human twins. | | | |
| | | | | | | Chem Senses | | | |
| | | | | | | 37: 869. | | | |
| rs12226920 | 12 | 11150046 | G | T | 22977065 | Knaapila, et al. | TAS2R20 | Bitter: | |
| | | | | | | 2012. Genetic | | Quinine | |
| | | | | | | analysis of | | | |
| | | | | | | chemosensory traits | | | |
| | | | | | | in human twins. | | | |
| | | | | | | Chem Senses | | | |
| | | | | | | 37: 869. | | | |
| rs1524600 | 7 | 80138303 | G | A | 22977065 | Knaapila, et al. | GNAT3 | Bitter: Cilantro pleasantness | |
| | | | | | | 2012. Genetic | | | |
| | | | | | | analysis of | | | |
| | | | | | | chemosensory traits | | | |
| | | | | | | in human twins. | | | |
| | | | | | | Chem Senses | | | |
| | | | | | | 37: 869. | | | |
| rs61729907 | 19 | 9325252 | G | A | 22977065 | Knaapila, et al. | ORD74 | Alcohol | Ethanol burn |
| | | | | | | 2012. Genetic | | trends: | |
| | | | | | | analysis of | | Ethanol | |
| | | | | | | chemosensory traits | | burn | |
| | | | | | | in human twins. | | | |
| | | | | | | Chem Senses | | | |
| | | | | | | 37: 869. | | | |
| rs1548803 | 12 | 10959031 | T | C | 22977065 | Knaapila, et al. | TAS2R38 | Bitter: | |
| | | | | | | 2012. Genetic | | Quinine | |
| | | | | | | analysis of | | | |
| | | | | | | chemosensory traits | | | |
| | | | | | | in human twins. | | | |
| | | | | | | Chem Senses | | | |
| | | | | | | 37: 869. | | | |
| rs4684677 | 3 | 10328453 | T | A | 21448464 | Landgren, et al. | GHRL | Sweet: Sucrose intake and | |
| | | | | | | 2011. PLos One. | | alcohol consumption | |
| | | | | | | 6: e18170 | | | |
| rs42451 | 3 | 10330377 | C | T | 21448465 | Landgren, et al. | GHRL | Sweet: Sucrose intake and | |
| | | | | | | 2011. PLos One. | | alcohol consumption | |
| | | | | | | 6: e18171 | | | |
| rs35680 | 3 | 10330564 | T | C | 21448466 | Landgren, et al. | GHRL | Sweet: Sucrose intake and | |
| | | | | | | 2011. PLos One. | | alcohol consumption | |
| | | | | | | 6: e18172 | | | |
| rs34911341 | 3 | 10331519 | C | T | 21448467 | Landgren, et al. | GHRL | Sweet: Sucrose intake and | |
| | | | | | | 2011. PLos One. | | alcohol consumption | |
| | | | | | | 6: e18173 | | | |
| rs696217 | 3 | 10331457 | G | T | 21448468 | Landgren, et al. | GHRL | Sweet: Sucrose intake and | |
| | | | | | | 2011. PLos One. | | alcohol consumption | |
| | | | | | | 6: e18174 | | | |
| rs26802 | 3 | 10332365 | T | G | 21448469 | Landgren, et al. | GHRL | Sweet: Sucrose intake and | |
| | | | | | | 2011. PLos One. | | alcohol consumption | |
| | | | | | | 6: e18175 | | | |
| rs2708377 | 12 | 11216315 | T | C | 23966204 | Ledda, et al. 2014. | TAS2R46 | Bitter: | Less intense |
| | | | | | | GWASofhumanbitter | | Caffeine/ | bitterness |
| | | | | | | taste perception | | Coffee | perception |
| | | | | | | identifiesnew | | | |
| | | | | | | loci and reveals | | | |
| | | | | | | additional complexity | | | |
| | | | | | | of bitter taste | | | |
| | | | | | | genetics | | | |
| rs28757581 | 6 | 29080004 | A | G | 22714804 | McRae, et al. 2012. | OR2J3 | Odor: cis-3-hexen-1-ol | |
| | | | | | | Genetic variation in | | (grassy odor) detection | |
| | | | | | | the odorant receptor | | | |
| | | | | | | OR2J3 is associated | | | |
| | | | | | | with the ability to | | | |
| | | | | | | detect the “grassy” | | | |
| | | | | | | smelling odor, cis-3- | | | |
| | | | | | | hexen-1-ol | | | |
| rs35744813 | 1 | 1265460 | C | T | 22546773 | Mennella, et al. | TAS1R3 | Sweet: | Reduced ability to |
| | | | | | | 2012. The proof is in | | Sucrose | detect sucrose, |
| | | | | | | the pudding: children | | preference | therefore increased |
| | | | | | | prefer lower fat but | | | sucrose preference |
| | | | | | | higher sugar than do | | | |
| | | | | | | mothers. | | | |
| rs4920566 | 1 | 19179824 | G | A | 22888812 | Piratsu, et al. 2012. | TAS1R2 | Alcohol | White wine liking |
| | | | | | | Genetics of food | | trends: | (P = 4.0 × 10−4) |
| | | | | | | preferences: a first | | Wine liking | |
| | | | | | | view from silk road | | | |
| | | | | | | populations. J Food | | | |
| | | | | | | Sci, 77: S413. | | | |
| rs3935570 | 1 | 19167371 | G | T | 22888812 | Piratsu, et al. 2012. | TAS1R2 | Alcohol | White wine liking |
| | | | | | | Genetics of food | | trends: | (P = 4.0 × 10−4) |
| | | | | | | preferences: a first | | Wine liking | |
| | | | | | | view from silk road | | | |
| | | | | | | populations. J Food | | | |
| | | | | | | Sci, 77: S413. | | | |
| rs2277675 | 17 | 3500510 | T | C | 22888812 | Piratsu, et al. 2012. | TRPV1 | Beet liking | |
| | | | | | | Genetics of food | | | |
| | | | | | | preferences: a first | | | |
| | | | | | | view from silk road | | | |
| | | | | | | populations. J Food | | | |
| | | | | | | Sci, 77: S413. | | | |
| rs71443637 | 12 | 11244194 | C | T | 24647340 | Piratsu, et al. 2014. | TAS2R43 | Bitter: | Increased coffee |
| | | | | | | Association Analysis | | Caffeine/ | liking (close to |
| | | | | | | of Bitter Receptor | | Coffee | significance) |
| | | | | | | Genes in Five | | | |
| | | | | | | Isolated Populations | | | |
| | | | | | | Identifies a | | | |
| | | | | | | Significant | | | |
| | | | | | | Correlation | | | |
| | | | | | | between TAS2R43 | | | |
| | | | | | | Variants and Coffee | | | |
| | | | | | | Liking | | | |
| rs9276975 | 6 | 32973599 | C | T | 25758996 | Piratsu, et al. 2015. | HLA-DOA | Alcohol | White wine liking |
| | | | | | | Genome-wide | | trends: | (p = 2.1 × 10−8), |
| | | | | | | association analysis | | Wine liking | Red wine liking |
| | | | | | | on five isolated | | | (p = 8.3 × 10−6) |
| | | | | | | populations identifies | | | |
| | | | | | | variants of the HLA- | | | |
| | | | | | | DOA gene | | | |
| | | | | | | associated with | | | |
| | | | | | | white wine liking. Eur | | | |
| | | | | | | J Hum Genet Pub | | | |
| | | | | | | online Mar. 11, | | | |
| | | | | | | 2015 | | | |
| rs34160967 | 1 | 6635306 | G | A | 19696921 | Shigemura, et al. | TAS1R1 | Umami: | more sensitive |
| | | | | | | 2009. PLoS One. | | perception | unami receptor |
| | | | | | | 4: e6717. | | | |
| rs307377 | 1 | 1269554 | C | T | 19696921 | Shigemura, et al. | TAS1R3 | Umami: | more sensitive |
| | | | | | | 2009. PLoS One. | | perception | unami receptor |
| | | | | | | 4: e6717. | | | |
| rs713598 | 7 | 141673345 | G | C | 17250611 | Wang, et al. 2007. | TAS2R38 | Alcohol | Higher mean |
| | | | | | | Alcoholism: Clinical | | trends: | Maxdrinks scores |
| | | | | | | and Experimental | | consumption | in AA but not EA |
| | | | | | | Research. 31: 209. | | | |
| rs1726866 | 7 | 141672705 | G | A | 17250611 | Wang, et al. 2007. | TAS2R38 | Alcohol | Higher mean |
| | | | | | | Alcoholism: Clinical | | trends: | Maxdrinks scores |
| | | | | | | and Experimental | | consumption | in AA but not EA |
| | | | | | | Research. 31: 209. | | | |
| rs10246939 | 7 | 141672604 | C | T | 17250611 | Wang, et al. 2007. | TAS2R38 | Alcohol | Higher mean |
| | | | | | | Alcoholism: Clinical | | trends: | Maxdrinks scores |
| | | | | | | and Experimental | | consumption | in AA but not EA |
| | | | | | | Research. 31: 209. | | | |
| rs846664 | 7 | 122635173 | G | T | 16051168 | Soranzo, et al. 2005. | TAS2R16 | Bitter: | Increased |
| | | | | | | Positive selection on | | Sensitivity | sensitivity to |
| | | | | | | a high-sensitivity | | to salicin, | salicin, arbutin, and |
| | | | | | | allele of the human | | arbutin, and | amygdalin |
| | | | | | | bitter taste receptor | | amygdalin | |
| | | | | | | TAS2R16 | | | |

urvey data was encoded numerically, with taste preferences encoded as −1 to +1, representing “dislike”, “no preference”, and “like”. Other questions were similarly encoded. Genotypes were encoded as 0, 1, or 2 for the minor homozygous, heterozygous, and major homozygous alleles, respectively. Participants were randomly assigned to a discovery and validation cohort, with ⅔ of participants being in the discovery cohort, and ⅓ in the validation cohort.

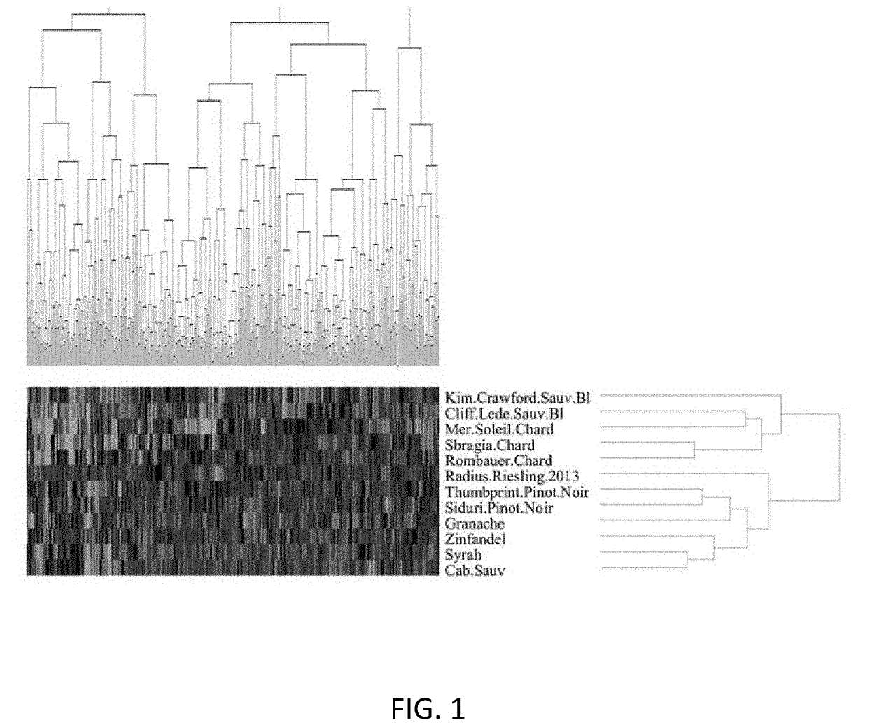

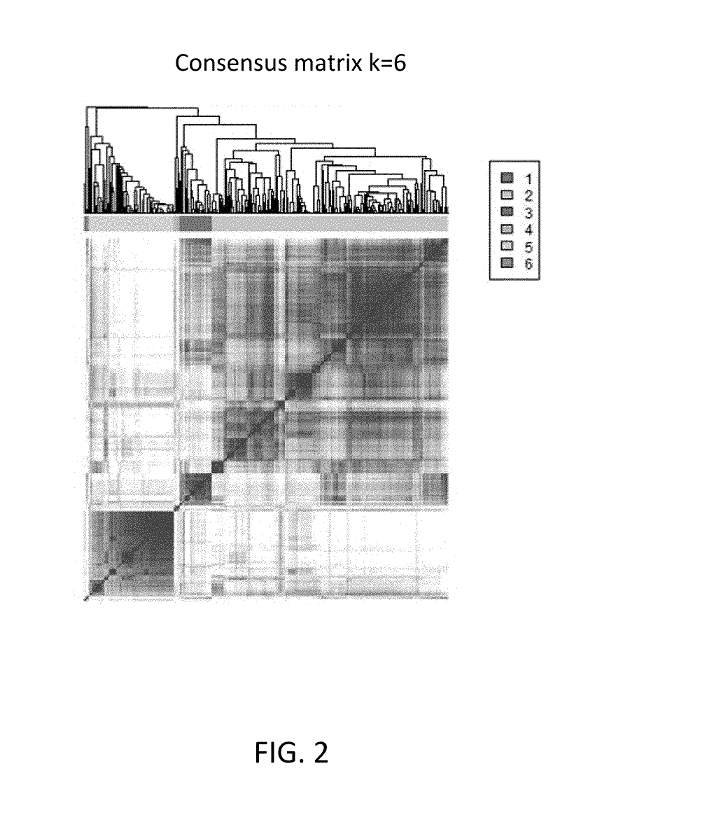

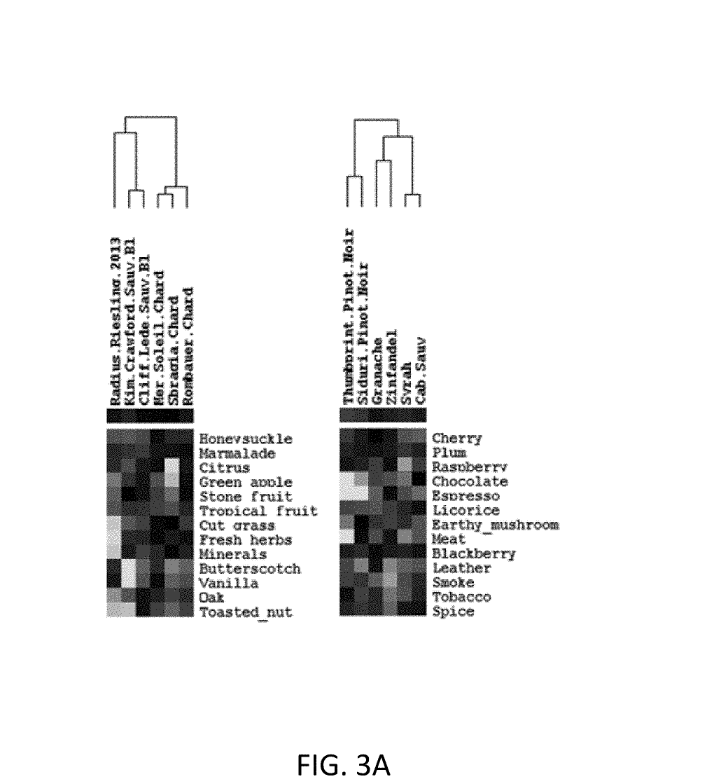

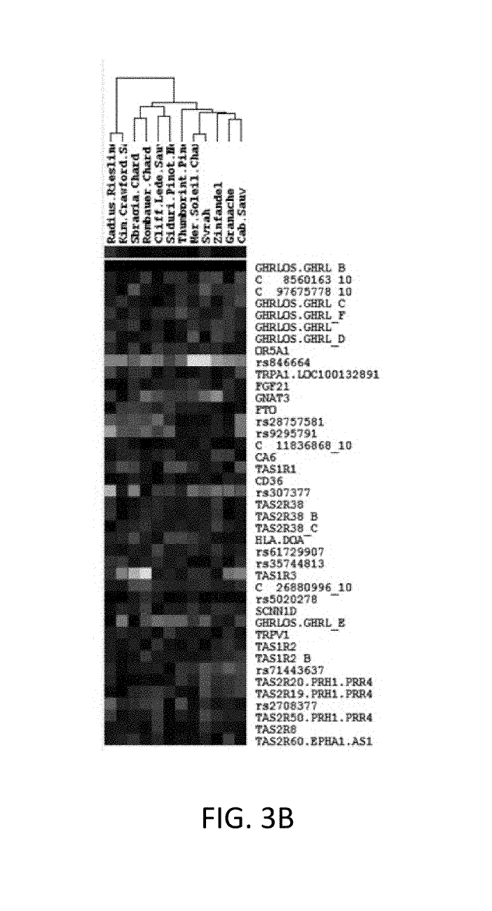

Initial exploration of the relationship between wine preferences was assessed using hierarchical and k-means clustering (FIG. 1). Relationships between wine preferences was also gauged using consensus clustering (FIG. 2), which allowed the determination of a reduced number of dimensions which could be used to describe wine preference. Association between wine preferences and survey responses and genotype was also explored using hierarchical clustering (FIGS. 3A, 3B) and assessed using ordered logistic regression.

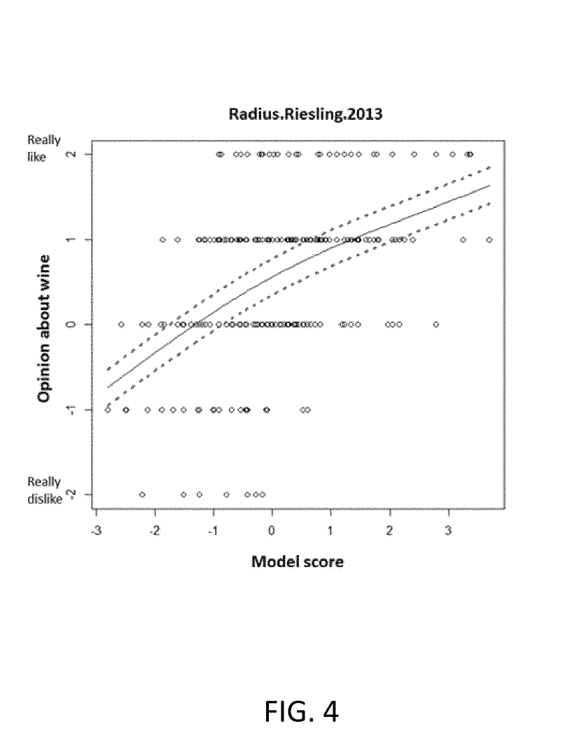

Variables for models predicting preference for the twelve wines were chosen from those survey responses and genetic variants having the greatest effect size (with any of the wines) and for which the 95% confidence intervals were both positive or both negative. Elastic net regression is used to remove variables not significantly independent of other variables. For the final model, thirteen survey responses and 5 genetic variants (Table 2) were selected and used to model each of the twelve wines (FIG. 4). Using the same variable for each typifying wine allows the score given to each to be comparable to the other wines. The effect size from the ordered logistic regression was used as the coefficient for the variables in the wine preference models.

TABLE 2
Coefficients Associated with Model Questions and Variants

| | Sweet | | Taste | | Wine | Drinks | | | | |
|---|---|---|---|---|---|---|---|---|---|---|
| | vs. | Taste | Pref. | | Knowledge | Wine | Taste | Taste | Wine | Taste |
| | Oak | Pref. | Black | Gender | Tries | Freq. | Pref. | Pref. | Knowledge | Pref. |
| | Response | Mushrooms | Coffee | Response | New | Response | Savory | Grass | Learning | Peaches |
| Radius | −0.7070 | −0.3674 | −0.0040 | 0.2200 | 0.0610 | −0.5627 | 0.6159 | 0.2581 | 0.8209 | 0.9655 |
| Riesling | | | | | | | | | | |
| 2013 | | | | | | | | | | |
| Kim | 0.1598 | 0.5625 | 0.2319 | −0.0846 | 0.4667 | 0.2691 | 0.5467 | 0.2350 | 0.1694 | 0.2174 |
| Crawford | | | | | | | | | | |
| Sauv | | | | | | | | | | |
| Bl | | | | | | | | | | |
| Cliff | 0.3616 | 0.4960 | 0.2617 | −0.5147 | 0.2775 | 0.6326 | 0.3980 | 0.3966 | −0.3697 | 0.0507 |
| Lede | | | | | | | | | | |
| Sauv | | | | | | | | | | |
| Bl | | | | | | | | | | |
| Mer | 0.3621 | 0.1043 | 0.2325 | −0.6683 | −0.0689 | 0.1524 | 0.1445 | 0.1194 | 0.0514 | 0.1733 |
| Soleil | | | | | | | | | | |
| Chard | | | | | | | | | | |
| Sbragia | 0.7345 | 0.1975 | 0.1921 | −0.3180 | 0.2836 | 0.2816 | −0.1413 | 0.1266 | −0.0027 | −0.3447 |
| Chard | | | | | | | | | | |
| Rombauer | 0.7313 | 0.4120 | 0.2476 | −0.5965 | 0.1898 | 0.3372 | 0.1399 | 0.1094 | 0.1285 | −0.1260 |
| Chard | | | | | | | | | | |
| Thumbprint | 0.6101 | 0.3863 | 0.3890 | −0.4984 | 0.5692 | 0.5367 | 0.7187 | 0.1918 | −0.2174 | 0.1889 |
| Pinot | | | | | | | | | | |
| Noir | | | | | | | | | | |
| Siduri | 0.4511 | 0.2911 | 0.4865 | −0.3183 | 0.8014 | 0.2144 | 0.3938 | 0.2427 | 0.2594 | 0.0824 |
| Pinot | | | | | | | | | | |
| Noir | | | | | | | | | | |
| Grenache | 0.7502 | 0.0817 | 0.2614 | −0.0860 | 0.6897 | 0.4433 | 0.2780 | 0.2058 | −0.4170 | 0.1805 |
| Zinfandel | 0.6424 | 0.0344 | 0.3501 | −0.1499 | 0.6028 | 0.1956 | 0.3077 | −0.0243 | −0.2566 | −0.2748 |
| Syrah | 0.8241 | 0.1071 | 0.4049 | −0.3238 | 0.5602 | 0.6074 | 0.1244 | −0.0673 | 0.0730 | 0.1045 |
| Cab | 0.7984 | 0.1887 | 0.7024 | −0.6284 | 0.3956 | 0.5565 | 0.4108 | 0.2052 | −0.1702 | −0.0939 |
| Sauv | | | | | | | | | | |

| | Taste | | | | | | | |
|---|---|---|---|---|---|---|---|---|
| | Pref. | Cheese | Taste | | | | | |
| | Sweet | Pref. | Pref. | | | | | |
| | Coffee | Swiss | Blackberries | rs3930459 | rs42451 | rs2708377 | rs71443637 | rs12226920 |
| Radius | 0.3143 | 0.377649 | −0.13437 | 0.00 | 0.00 | 0.00 | 0.00 | 0.00 |
| Riesling | | | | | | | | |
| 2013 | | | | | | | | |
| Kim | −0.1746 | 0.405738 | 0.244817 | 0.00 | 0.00 | 0.00 | 0.00 | 0.00 |
| Crawford | | | | | | | | |
| Sauv | | | | | | | | |
| Bl | | | | | | | | |
| Cliff | −0.2990 | −0.06705 | −0.04484 | 0.00 | 0.00 | 0.00 | 0.00 | 0.00 |
| Lede | | | | | | | | |
| Sauv | | | | | | | | |
| Bl | | | | | | | | |
| Mer | −0.1551 | 0.285404 | 0.157368 | 0.00 | 0.45 | 0.00 | 0.00 | 0.00 |
| Soleil | | | | | | | | |
| Chard | | | | | | | | |
| Sbragia | −0.1186 | 0.498293 | 0.280204 | 0.40 | 0.00 | 0.00 | 0.00 | 0.00 |
| Chard | | | | | | | | |
| Rombauer | −0.1122 | 0.314874 | 0.059932 | 0.43 | 0.00 | 0.00 | 0.00 | 0.00 |
| Chard | | | | | | | | |
| Thumbprint | 0.0729 | −0.13314 | 0.688492 | 0.00 | 0.00 | 0.40 | 0.00 | −0.28 |
| Pinot | | | | | | | | |
| Noir | | | | | | | | |
| Siduri | −0.2415 | 0.429498 | 0.65768 | 0.00 | 0.00 | 0.00 | 0.00 | 0.00 |
| Pinot | | | | | | | | |
| Noir | | | | | | | | |
| Grenache | −0.0464 | −0.447 | 0.783813 | 0.00 | 0.00 | 0.00 | 0.49 | −0.27 |
| Zinfandel | −0.3745 | −0.6555 | 0.322897 | 0.00 | 0.00 | 0.00 | 0.34 | 0.00 |
| Syrah | −0.3109 | −0.45071 | 0.875547 | 0.00 | 0.00 | 0.00 | 0.43 | −0.41 |
| Cab | −0.3482 | −0.36389 | 0.973206 | 0.00 | 0.00 | 0.00 | 0.37 | −0.33 |
| Sauv | | | | | | | | |

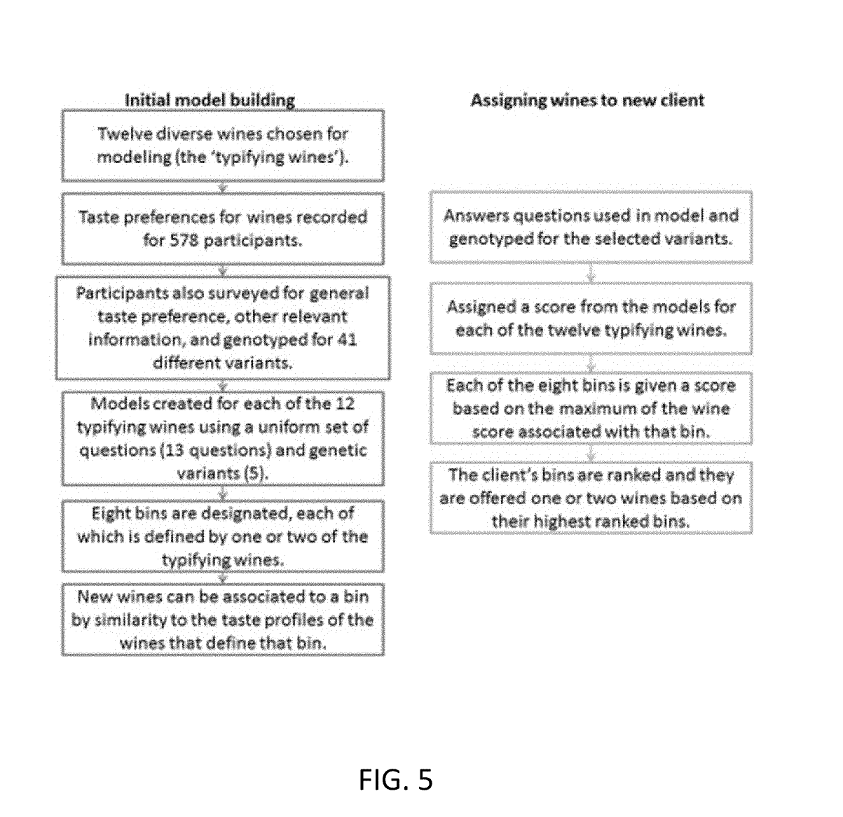

The basic process via which subjects are offered a wine is shown in FIG. 5. In short, using the models for twelve diverse wines established with the initial study population, subjects are given a score for the twelve wines used to describe a wide variant of wine preferences, and then given a score for a set of eight wine ‘bins’ that describe broad categories of wine preferences. The wine bin scores are ranked, and bins that are likely to be preferred will have higher scores and less preferred bins.

Wines were grouped according to eight bins defined by hierarchical clustering (Table 3), and a person's score for each bin was equal to that given by the model for the wine associated with that bin which had the highest score. For example, if an individual's score for Grenache was 1.2 and for the Zinfandel was 2.4, the individual's score for the “lighter chocolaty raspberry reds” bin, which those two wines are associated with, would be 2.4. Association of individual bin scores with actual averaged preference for the wines assigned to the bin was measured using ordered logistic regression (Table 4). All models were found to be highly significant.

Wines are assigned to the subject by ranking the bin scores, and offering the subject a wine from the highest ranked bin. If the other bins have negative rankings, no other wines are offered. If the other bins are positive, that is, the subject has a higher than average preference for wines of those bins, then another wine is offered. If the lowest ranked bin of the same wine color (i.e. red or white) as the highest ranked bin is greater than the highest ranked bin of the opposite color, then a wine of the second highest ranked bin is offered. Otherwise, a wine of the highest ranked bin of the opposite color is offered.

These results showed that methods and systems of the invention are useful for predicting wine preferences in a subject. These results further suggested that the methods and systems of the present invention are useful for creating a taste profile for a subject and providing a wine to a subject based on the subject's taste profile.

TABLE 3
Wine Models and Their Association with Bins.

| bin | typifying wine |
|---|---|
| sweet whites | Radius Riesling 2013 |
| crisp and citrusy whites | Kim Crawford Sauv Bl |
| crisp and floral whites | Cliff Lede Sauv Bl |
| unoaked full-bodied whites | Mer Soleil Chard |
| oaked full-bodied whites | Sbragia Chard |
| | Rombauer Chard |
| light, cherry reds | Thumbprint Pinot Noir |
| | Siduri Pinot Noir |
| light chocolaty raspberry reds | Grenache |
| | Zinfandel |
| bold smoky blackberry reds | Syrah |
| | Cab Sauv |

TABLE 4
Testing of Predicted Bin Preferences.

| | |
|---|---|
| effect (95% CI) | P value |

| | | | |
|---|---|---|---|
| sweet whites | 0.52 | (0.31, 0.73) | 1.55E−06 |
| crisp and citrusy whites | 0.43 | (0.2, 0.66) | 0.000321 |
| crisp and floral whites | 0.61 | (0.39, 0.83) | 4.42E−08 |
| unoaked full-bodied whites | 0.58 | (0.25, 0.91) | 0.00067 |
| oaked full-bodied whites | 0.69 | (0.41, 0.97) | 1.21E−06 |
| light, cherry reds | 0.7 | (0.5, 0.91) | 2.37E−11 |
| light chocolaty raspberry reds | 0.58 | (0.39, 0.78) | 6.99E−09 |
| bold smoky blackberry reds | 0.61 | (0.44, 0.79) | 2.96E−12 |

While the preferred embodiments of the invention have been illustrated and described, it will be appreciated that various changes can be made therein without departing from the spirit and scope of the invention.

What is claimed is:

1. A method for assaying a sample from a subject, the method comprising: a) obtaining a biological sample comprising nucleic acids from a subject; b) processing the sample to isolate or enrich the sample for the nucleic acids; and c) assaying a plurality of single nucleotide polymorphisms (SNPs) in the sample, wherein the plurality of SNPs comprises one or more SNPs selected from Table 1.

2. The method of claim 1, further comprising obtaining taste preference information from the subject.

3. The method of claim 1, wherein the plurality of SNPs comprises at least 5, 10, 15, 20, 25, 30, 35 or 41 SNPs selected from Table 1.

4. The method of claim 1, wherein the one or more SNPs comprise rs838133, rs1421085, rs3930459, rs2274333, rs111615792, rs72921001, rs7792845, rs10772420, rs846672, rs9295791, rs6591536, rs1761667, rs10772397, rs586965, rs5020278, rs11988795, rs4595035, rs12226920, rs1524600, rs61729907, rs1548803, rs4684677, rs42451, rs35680, rs34911341, rs696217, rs26802, rs2708377, rs28757581, rs35744813, rs4920566, rs3935570, rs2277675, rs71443637, rs9276975, rs34160967, rs307377, rs713598, rs1726866, rs10246939, and rs846664.

5. A method for predicting wine preferences of a subject, the method comprising: a) providing a biological sample comprising DNA from the subject; b) genotyping a plurality of SNPs in the sample, wherein the plurality of SNPs comprises one or more SNPs selected from Table 1; c) obtaining taste preference information from the subject; and d) determining the subject's taste preference scores for one or more wines, thereby predicting wine preferences for the subject.

6. The method of claim 5, further comprising: calculating wine bin scores based on the genotyping from step (b), the information obtained about the subject from step (c), and the taste preference scores for the one or more wines from step (d).

7. The method of claim 6, wherein the wine bins comprise one or more wine bins selected from the group consisting of a first wine bin for sweet white wines, a second wine bin for crisp and citrusy white wines, a third wine bin for crisp and floral white wines, a fourth wine bin for unoaked full-bodied white wines, a fifth wine bin for oaked full-bodied white wines, a sixth wine bin for light cherry red wines, a seventh wine bin for light chocolaty raspberry red wines, and an eighth wine bin for bold smoky blackberry red wines.

8. The method of claim 6, wherein the wine bin scores are used to predict that the subject will prefer wines from a wine bin having a higher wine bin score than a wine bin having a lower wine bin score.

9. The method of claim 5, wherein the one or more SNPs comprise rs838133, rs1421085, rs3930459, rs2274333, rs111615792, rs72921001, rs7792845, rs10772420, rs846672, rs9295791, rs6591536, rs1761667, rs10772397, rs586965, rs5020278, rs11988795, rs4595035, rs12226920, rs1524600, rs61729907, rs1548803, rs4684677, rs42451, rs35680, rs34911341, rs696217, rs26802, rs2708377, rs28757581, rs35744813, rs4920566, rs3935570, rs2277675, rs71443637, rs9276975, rs34160967, rs307377, rs713598, rs1726866, rs10246939, and rs846664.

10. A method for providing a wine to a subject, the method comprising the following steps:
a) providing a biological sample comprising DNA from the subject;
b) genotyping the sample to determine which alleles are present at a plurality of single nucleotide polymorphisms, wherein the plurality of SNPs is selected from the group consisting of rs838133, rs1421085, rs3930459, rs2274333, rs111615792, rs72921001, rs7792845, rs10772420, rs846672, rs9295791, rs6591536, rs1761667, rs10772397, rs586965, rs5020278, rs11988795, rs4595035, rs12226920, rs1524600, rs61729907, rs1548803, rs4684677, rs42451, rs35680, rs34911341, rs696217, rs26802, rs2708377, rs28757581, rs35744813, rs4920566, rs3935570, rs2277675, rs71443637, rs9276975, rs34160967, rs307377, rs713598, rs1726866, rs10246939, and rs846664;
c) obtaining information about the subject;
d) determining the subject's taste preference scores for one or more wines comprising Riesling, Sauvignon Blanc, Chardonnay, Pinot Noir, Grenache, Zinfandel, Syrah, and Cabernet Sauvignon; and
e) calculating wine bin scores for one or more wine bins based on the genotyping from step (b), the information obtained about the subject from step (c), and the taste preference scores for one or more wines from step (d); and
f) providing the subject with at least one wine from the wine bin that has the subject's highest wine bin score.

11. The method of claim 10, wherein the plurality of SNPs comprises at least 5, 10, 15, 20, 25, 30, 35 or 41 SNPs selected from Table 1.

12. The method of claim 10, wherein the information about the subject is selected from the group consisting of the subject's gender; whether the subject prefers sweet wine or oaked wine; taste preferences of the subject for mushrooms, black coffee, savory flavor, grass, peaches, sweet coffee, Swiss cheese, and blackberries; interest in learning about wines; whether or not the subject tries new wines, and whether or not the subject drinks wine frequently.

13. The method of claim 10, wherein said wine bins comprise a first wine bin for sweet white wines, a second wine bin for crisp and citrusy white wines, a third wine bin for crisp and floral white wines, a fourth wine bin for unoaked full-bodied white wines, a fifth wine bin for oaked full-bodied white wines, a sixth wine bin for light cherry red wines, a seventh wine bin for light chocolaty raspberry red wines, and an eighth wine bin for bold smoky blackberry red wines.

14. The method of claim 10, further comprising providing said at least one wine to the subject on a periodic basis.

15. The method of claim 10, wherein genotyping the single nucleotide polymorphisms is performed by a method selected from the group consisting of dynamic allele-specific hybridization (DASH), microarray analysis, Tetra-primer ARMS-PCR, a TaqMan 5′-nuclease assay; an Invader assay with Flap endonuclease (FEN), a Serial Invasive Signal Amplification Reaction (SISAR), an oligonucleotide ligase assay, restriction fragment length polymorphism (RFLP), single-strand conformation polymorphism, temperature gradient gel electrophoresis (TGGE), denaturing high performance liquid chromatography (DHPLC), sequencing, and immunoassay.


SEQUENCE LISTING (5 sequences)

SEQ ID NO: 1 - DNA, 51 bp, Homo sapiens
  allele (26)..(26): n is C or T
  misc_feature (26)..(26): n is a, c, g, or t
agtcatcaagatgtagagtcatgaantgtacactttacatacattggattt

SEQ ID NO: 2 - DNA, 51 bp, Homo sapiens
  allele (26)..(26): n is C or T
  misc_feature (26)..(26): n is a, c, g, or t
agcgagcaggggctgggggagcttcngacggctttattcctcgactggaga

SEQ ID NO: 3 - DNA, 51 bp, Homo sapiens
  allele (26)..(26): n is A or G
  misc_feature (26)..(26): n is a, c, g, or t
aacaaacctttgcaatttcccatctntttttgcttttggctcctgaattcc

SEQ ID NO: 4 - DNA, 51 bp, Homo sapiens
  allele (26)..(26): n is C or T
  misc_feature (26)..(26): n is a, c, g, or t
gctgggatcttgagatcctttaccanggagctgcatcttcttgagatgttt

SEQ ID NO: 5 - DNA, 51 bp, Homo sapiens
  allele (26)..(26): n is G or T
  misc_feature (26)..(26): n is a, c, g, or t
ttatatacgtgtgtttcatcacaagntgacaaaccaaaaagaacaaagacc